(19) United States
(12) Patent Application Publication
(10) Pub. No.: US 2025/0262482 A1
(43) Pub. Date: Aug. 21, 2025

(54) EXERCISE TOLERANCE USING AN IMPLANTABLE OR WEARABLE HEART MONITOR

(71) Applicant: Medtronic, Inc., Minneapolis, MN (US)

(72) Inventors: Ryan D. Wyszynski, Oak Grove, MN (US); Geert Morren, Vissenaken (BE); Hyun J. Yoon, Vadnais Heights, MN (US)

(21) Appl. No.: 18/857,295

(22) PCT Filed: Apr. 4, 2023
(86) PCT No.: PCT/IB2023/053412

§ 371 Date: Oct. 16, 2024

Related U.S. Application Data
(60) Provisional application No. 63/363,460, filed on Apr. 22, 2022.

(51) Int. Cl. A63B 24/00; A61B 5/00; A61B 5/0205; A61B 5/07; A61B 5/287; G16H 40/67

(52) U.S. Cl. CPC A63B 24/0075; A61B 5/0205; A61B 5/076; A61B 5/7275; G16H 40/67; A61B 5/287

(57) ABSTRACT

A device includes a memory and processing circuitry coupled to the memory and configured to: receive, from a medical monitoring device, a set of one or more patient parameters; determine an exercise tolerance level based on the set of one or more patient parameters and based on at least one comorbidity of the patient; determine that a present activity level for the patient exceeds the exercise tolerance level for the patient; and send a notification to the patient in response to determining that the present activity level exceeds the exercise tolerance level for the patient.

This application claims the benefit of U.S. Provisional Application Ser. No. 63/363,460, filed Apr. 22, 2022, the entire content of which is incorporated herein by reference.

### FIELD

The disclosure relates to medical devices and, more particularly, medical devices for detecting or monitoring heart conditions.

### BACKGROUND

A variety of medical devices have been used or proposed for use to deliver a therapy to and/or monitor a physiological condition of patients. As examples, such medical devices may deliver therapy and/or monitor conditions associated with the heart, muscle, nerve, brain, stomach or other organs or tissue. Medical devices that deliver therapy include medical devices that deliver one or both of electrical stimulation or a therapeutic agent to the patient. Some medical devices have been used or proposed for use to monitor cardiovascular diseases or to detect cardiovascular events.

Heart failure (HF) is the most common cardiovascular disease that causes significant economic burden, morbidity, and mortality. In the United States alone, roughly 5 million people have HF, accounting for a significant number of hospitalizations. HF may result in cardiac chamber dilation, increased pulmonary blood volume, and fluid retention in the lungs. Generally, the first indication that a physician has of HF in a patient is not until it becomes a physical manifestation with swelling or breathing difficulties so overwhelming as to be noticed by the patient who then proceeds to be examined by a physician. This is undesirable since hospitalization at such a time would likely be required for a cardiac heart failure patient to remove excess fluid and relieve symptoms.

### SUMMARY

According to the techniques of this disclosure, a medical device system may determine an exercise tolerance threshold based on patient parameters. A medical device may monitor one or more same or different parameters to determine a present activity level for the patient. In response to the patient activity level exceeding the exercise tolerance level, the medical device system may generate an alert to notify the patient to dangerous levels of activity or exercise. Additionally or alternatively, the system may also send to the patient a recommendation of specific behavior changes that may reduce a likelihood that the patient will experience a cardiac event. In this manner, the system may provide a clinical advantage by enabling a patient, without being directly observed by a clinician, to know their safe level of exercise and warn the patient if they are exceeding that safe level of exercise, which may help the patient avoid adverse health events.

In contrast to clinic visit/stay based monitoring of a patient's condition and risk of adverse health events, the techniques of this disclosure may be implemented by systems including implantable medical device (IMDs) and computing devices that can autonomously and continuously (e.g., on a periodic or triggered basis without human intervention) collect physiological parameter data while the implantable medical device is subcutaneously implanted in a patient over months or years and perform numerous operations per second on the data to enable the systems herein to determine varying risk levels of the cardiac event and associated exercise tolerance threshold. Using techniques of this disclosure with an IMD may be advantageous when a physician cannot be continuously present with the patient over weeks or months to evaluate the physiological parameters and/or where performing the operations on the data described herein (e.g., application of a probability model) could not practically be performed in the mind of a physician. Using the techniques of this disclosure with autonomously/continuously operating IMDs and computing devices may provide a clinical advantage in timely detecting changes in a patient's condition and exercise tolerance threshold and providing timely alerts to the patient and/or caregiver.

In some examples, the techniques and systems of this disclosure may use a probability model to more accurately determine exercise tolerance thresholds based on physiological data. In some examples, the probability model is trained with a set of training instances, where one or more of the training instances comprise data that indicate relationships between various input data and cardiac risk levels. Because the probability model is trained with potentially thousands or millions of training instances, the probability model may reduce the amount of error in determining cardiac risk and, consequently exercise tolerance level, which may improve patient outcomes.

According to one example of this disclosure, a device includes a memory and processing circuitry coupled to the memory and configured to: receive, from a medical monitoring device, a set of one or more patient parameters; determine an exercise tolerance level based on the set of one or more patient parameters and based on at least one comorbidity of the patient; determine that a present activity level for the patient exceeds the exercise tolerance level for the patient; and send a notification to the patient in response to determining that the present activity level exceeds the exercise tolerance level for the patient.

According to another example of this disclosure, a device includes a memory and processing circuitry coupled to the memory and configured to: receive, from a medical monitoring device, a set of one or more patient parameters; determine an exercise tolerance level based on the set of one or more patient parameters and based on at least one comorbidity of the patient; and transmit the exercise tolerance level to a patient device.

According to another example of this disclosure, a method includes receiving, from a medical monitoring device, a set of one or more patient parameters; determining an exercise tolerance level based on the set of one or more patient parameters and based on at least one comorbidity of the patient; determining that a present activity level for the patient exceeds the exercise tolerance level for the patient; and sending a notification to the patient in response to determining that the present activity level exceeds the exercise tolerance level for the patient.

The summary is intended to provide an overview of the subject matter described in this disclosure. It is not intended to provide an exclusive or exhaustive explanation of the systems, device, and methods described in detail within the accompanying drawings and description herein. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 is an example diagram of a probability framework including evidence nodes from diagnostic states of various parameters and one parent node.

FIG. 2 is a block diagram illustrating an example system that includes medical device(s) used to obtain diagnostic states from the various parameters for use as evidence nodes.

FIG. 3 is a functional block diagram illustrating an example configuration of the external device of FIG. 2.

FIG. 4 is a functional block diagram illustrating an example framework for a probability model to determine health risk probabilities for a patient using evidence obtained from the system of FIG. 2.

FIG. 5 illustrates the environment of an example medical system in conjunction with the patient, including an example implantable medical device (IMD) used to determine parameters of the patient.

FIG. 6 is a functional block diagram illustrating an example configuration of an IMD of FIG. 5.

FIG. 7 is a conceptual side-view diagram illustrating an example IMD of the medical system of FIGS. 5 and 6 in greater detail.

FIG. 8 is a flow diagram illustrating a process in accordance with the techniques described herein.

FIG. 9A is a perspective drawing illustrating an example IMD.

FIG. 9B is a perspective drawing illustrating another example IMD.

Like reference characters denote like elements throughout the description and figures.

### DETAILED DESCRIPTION

Patients with implantable monitors (e.g., the Reveal LINQ™ or LINQ II™ insertable cardiac monitors, available from Medtronic, Inc.) typically also have other comorbidities such as coronary artery disease (CAD) and can be at risk for exercise induced arrhythmias, exercise induced heart attacks, and other cardiac events. Patients with heart failure, CAD, with arrhythmias, post myocardial infarction (MI), or post heart surgery may need to understand the limits on their safe levels of exercise, which can differ for different patients. This disclosure describes techniques for an early warning system for these patients that may prevent or mitigate adverse events, such as an arrhythmia, MI, or ischemic stroke. According to techniques of this disclosure, a medical device system may be configured to provide a patient with an indication of a predicted safe level of activity, which may also be referred to as an exercise tolerance. As will be discussed in more detail in this disclosure, the exercise tolerance may be patient-specific in that it is determined based on continually, asynchronously, or periodically monitored patient parameters, and may further be based on any known comorbidities for the patient. Moreover, the exercise tolerance may be dynamic in the sense that it changes, as a patient's condition changes.

According to the techniques of this disclosure, the medical device system may determine the exercise tolerance based on any one or more of the following parameters: whether the patient has coronary artery disease, prior MI, heart rate, heart rate recovery time post exercise, respiration (rate and/or volume), oxygen saturation, PTT (pulse wave transit time-measured as the time from the R-wave of the ECG to the arrival of the pulse wave at a blood flow sensor, such as an oxygen saturation sensor), EGM morphology, EMG (muscle activity), ST Segment deviation (e.g., depression or elevation), T-wave alternans, and activity.

The medical device may monitor one or more of the above parameters and based on one or more of the above parameters may also determine a present activity level for the patient. In response to the patient activity level exceeding the exercise tolerance level, the medical device system may generate an alert to notify the patient to dangerous levels of activity or exercise. The exercise tolerance level may be set, by a clinician, based on any one or more of the parameters above and the patient's exercise capacity characteristics. Once the patient's present activity level exceeds the exercise tolerance level, the medical device system may send an alert to the patient to warn them of dangerous activity levels. Additionally or alternatively, the system may also send to the patient a recommendation of specific behavior changes that may reduce a likelihood that the patient will experience a cardiac event. In this manner, the system may enable a patient to know their safe level of exercise and warn the patient if they are exceeding that safe level of exercise, which may help the patient avoid adverse health events.

The medical device system may collect the following data throughout the day. The medical device system may input the data into a Bayesian Network (or other suitable machine learning or artificial intelligence model). Each parameter may be weighted to determine if the patient should be alerted that they are at a dangerous activity level.

In one example implementation, the medical device system may include a medical monitoring device (either wearable or implantable), a user device such as a phone, and a cloud-based computing system. The medical monitoring device may collect real-time parameter data (e.g., HR, O2 Saturation, respiration, ST segment deviation, EGM morphology, PTT) and obtain the patient's medical history (e.g., CAD, prior MI, prior arrhythmia, results of an exercise ECG test, etc.) from the cloud-computing device. The cloud-computing device or the user device may implement an algorithm (e.g., the Bayesian Network) to determine the exercise tolerance level. Upon a trigger detected by the medical monitoring device, the medical monitoring device sends the collected data to the user device. The user device may receive a portion or all of the data and determine if the patient is at a dangerous activity level based on a determination of whether or not the patient's present activity level exceeds the exercise tolerance level. The user device may alert the patient that the patient is at risk of an adverse cardiac event due to their present activity level and provide recommended behavior changes to reduce that risk.

In some examples, the techniques of this disclosure may be performed in part using an implantable medical device configured to continuously monitor certain patient parameters, which may provide monitoring during periods of time that external devices often do not. As one example, patients typically do not wear external devices overnight or early in the morning, but transitioning from a sleep state to a wake state can be a particularly dangerous time for some patients due to rapid changes in the sympathetic nervous system. Moreover, certain patient parameters indicative of the patient's health status that tend to present overnight may not be identified by certain external devices. Accordingly, by using an internal (implanted) medical device, the techniques of this disclosure may provide a more accurate exercise tolerance for a patient and may also provide monitoring during time periods that other devices may miss.

In some examples, the prediction and/or probability modeling described herein for determining a patient-specific exercise threshold may include Bayesian Belief Networks (BBN) or Bayesian machine learning (ML) models (these sometimes referred to as Bayesian Networks or Bayesian frameworks herein), Markov random fields, graphical models, artificial intelligence (AI) models (e.g., Naive Bayes classifiers), and/or other belief networks, such as sigmoid belief networks, deep belief networks (DBNs), etc. In other examples, the disclosed technology may leverage non-Bayesian prediction or probability modeling, such as frequentist inference modeling or other statistical models. In addition, known model selection-techniques, such as Bayesian information criterion (BIC) or Akaike information criterion (AIC), may be used to evaluate probability models prior to use.

In some examples, an integrated diagnostics model may be used to determine a number of criteria that are met based on sensed or measured parameters. For example, the probability model may determine that X of Y criteria have been met with respect to the parameters. In such examples, Y may be the maximum number of criteria possible given the particular configuration of parameters the probability model is using, and X may be a variable less than or equal to Y that increments based on the parameters meeting certain criteria. In an illustrative example, the probability model may increment X in response to determining that the patient has a high heart rate indicating a high diagnostic state.

Processing circuitry may determine, from the respective parameter values, diagnostic states for each parameter. Processing circuitry may compare the heart rate score to one or more risk thresholds to determine a diagnostic state of high (H) risk, medium (M) risk, or low (L) risk, in some examples. In some examples, processing circuitry may determine a joint diagnostic state based on multiple parameters that are independent of one another.

In some examples, diagnostic states may include a finite number of potential diagnostic states for each parameter (e.g., very high, high, medium, low, very low, etc.). For example, the diagnostic states may include states of high risk, medium risk, or low risk, for each parameter. In some instances, one or more of the parameters can have a different number of potential diagnostic states (e.g., one state, two states, three states, or more), whereas other parameters may have a greater or lesser number of potential diagnostic states. For example, heart rate may have three diagnostic states (H, M, and L), whereas prior arrhythmia may have less than three diagnostic states (H and L). In other examples, diagnostic states may include a continuum or sliding spectrum of diagnostic state values, rather than discrete states.

Diagnostic states of the parameters may be independent for each parameter. For example, a diagnostic state for a first set of one or more parameters may be independent of diagnostic states associated with one or more other parameters. In some examples, the probability mode framework, such as a BBN framework, may include additional parameters, where the respective values of the parameters are conditionally independent of one another. In an example, a high night heart rate may indicate a worsening condition. In addition, a decrease in O2 saturation may indicate a hypoxemia. In such examples, each of these parameters may provide indications of a dangerous activity level. In accordance with techniques of this disclosure, however, each of these conditionally independent parameters may provide stronger evidence when used together to predict an adverse health event resulting from the dangerous activity level.

In an illustrative example, two example parameters may be conditionally independent of one another in the absence of an adverse health event. To illustrate, in the presence of the adverse health event, such as a myocardial ischemia, the two example parameter variables may be correlated or dependent on one another, but in the absence of the adverse health event, the two variables may change independently from one another, indicating that the two variables are conditionally independent of one another. For example, in some instances, respiration rate (RR) will increase, whereas subcutaneous impedance will decrease during an adverse health event, such as HF, because HF may be the cause of these changes but not an effect of such changes. For example, an increase in RR does not cause HF, whereas RR may increase as a result of HF. In another example, RR may increase when a patient has anemia, whereas subcutaneous impedance may not change either way as a result of the anemia.

In some examples, processing circuitry may identify diagnostic states for parameters that the processing circuitry has deemed relevant to the goal of the probability model. In a non-limiting example, if the goal is to determine an exercise threshold that if exceeded may have a relatively high likelihood of causing a clinically-significant cardiac event (e.g., meriting a remote-care phone call or clinic visit or hospital admission), certain parameters are more relevant to that probability determination than others.

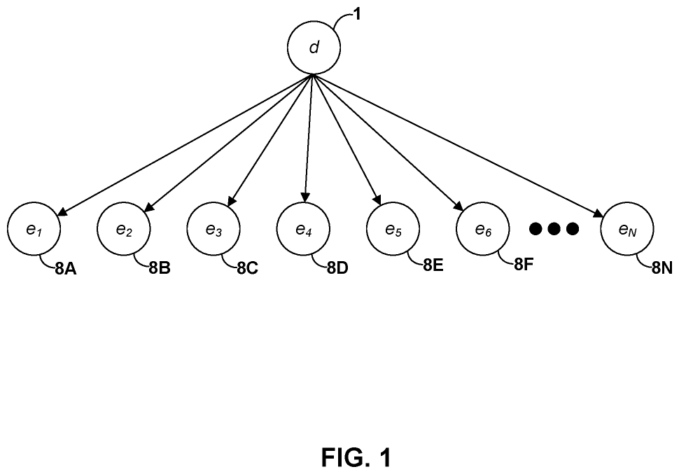

The diagnostic states may serve as evidence nodes for the probability model. FIG. 1 represents an example probability model framework that includes a parent node 1 and a plurality of evidence nodes 8A-8N (collectively, “evidence nodes 8”). Parent node 1 represents the posterior probability (e.g., the probability that an adverse health event is to occur at a certain activity level based on diagnostic states of evidence nodes 8). In an example, the adverse health event may include a cardiac event occurring at a certain activity level, where d=H, for illustration purposes. The probability model may include any number of evidence nodes 8, as illustrated by evidence node 8N. Each of evidence nodes 8 may correspond to one or more parameters of a patient. As further discussed herein, each one of evidence nodes 8 may include a diagnostic state derived from one or more values that correspond to one or more parameters. In examples involving discrete states of d, the posterior probability for the occurrence of the cardiac event (d=H) may be expressed as:

$$P ( d = H \vert {e}_{1} , … , {e}_{N} ) = \frac{P ( d = H ) {\prod }_{i = 1}^{N} P ( {e}_{i} \vert d = H )}{{\sum }_{{d}_{1}} P ( d ) {\prod }_{i = 1}^{N} P ( {e}_{i} \vert d )}$$

In such examples, P(d) may represent a prior probability value, P(eᵢ|d) may represent a conditional likelihood parameter, d represents parent node 1, and e₁-e_{N} represent evidence nodes 8 in FIG. 1. Processing circuitry may determine the prior probability value and the conditional likelihood from existing parameter values prior to clinical event d in previous clinical study data. In some examples, the conditional likelihood parameter may assume, using previous probability data, what probability distribution is likely to exist, such that the processing circuitry can assume what probability scores are unlikely based on previous probability data. In some examples, the prior probability value may include a probability distribution absent any diagnostic states to use as evidence nodes. In other words, the prior probability value is what processing circuitry may believe at a particular point of time, whereas the posterior probability is what processing circuitry may believe in the presence of incoming diagnostic information.

In some examples, the probability score may include a joint probability distribution. In an example, for a n-node Bayesian network (where paᵢ is the parent node of node xi), the joint probability distribution may be expressed as:

$$P ( {x}_{i} , … , {x}_{N} ) = {\prod }_{i}^{N} P ( {x}_{i} \vert {pa}_{i} ) ,$$

For example, a posterior probability may involve determining joint probability distributions and defining multiple combinations of conditional probabilities. A probability model may provide a framework for assumptions regarding the explicit relationship between parameter values to make these determinations more feasible. For example, Bayesian theory may assign explicit relationships between parameter values in order to determine probability scores from the various evidence nodes 8 in FIG. 1. For example, a posterior probability may include a posterior distribution. In some cases, the posterior distribution may include a Gaussian distribution. In other cases, the posterior distribution may include a non-Gaussian distribution.

In some examples, processing circuitry may determine and/or utilize conditional likelihood tables, BBN tables, prior probability values, etc. For example, the conditional likelihood parameters may take the form of conditional likelihood tables defined for each diagnostic state for each parameter. The posterior probability may then be tabulated for all possible combinations of diagnostic states to determine a posterior probability, or in some instances, a probability table.

In some instances, a single evidence node may be derived from multiple parameters, such as with a Multi-Variable Node (MVN). In an example, MVNs may be based on multiple parameters, such as atrial fibrillation (AF) burden as a first parameter and ventricular rate values as a second parameter, where the parameters factor into a single evidence node.

Processing circuitry may use the evidence nodes as input to a probability model to determine a posterior probability score. In such instances, the posterior probability score indicates a likelihood that a patient will experience an adverse cardiac event at a certain activity level within a predetermined period of time (e.g., within 30 minutes of being at the certain activity level). As discussed herein, the probability model may use as additional inputs the prior probability value and a conditional likelihood parameter to determine the posterior probability score. In some examples, processing circuitry may then update the probability model using the determined posterior probability score.

Based on the probability score, an exercise tolerance threshold can be determined. For example, a system may determine probability scores for a plurality of activity levels, and the system, or a user of the system, may select an activity level that corresponds to an acceptable probability of risk for a patient. In some examples, the system may determine different exercise tolerance thresholds for different metrics. For example, an activity level based on a respiration rate for the patient may have a different exercise tolerance threshold than an activity level based on a heart rate. The exercise tolerance threshold may be implemented in various manners. As one example, the exercise tolerance threshold may be viewed as an activity level, above which causes a person's risk of an adverse cardiac to exceed a desirable risk level. In other examples, the exercise tolerance threshold may not correspond to a precise activity level, but instead may correspond to a certain risk level, deemed by the patient or a clinician to be undesirably high.

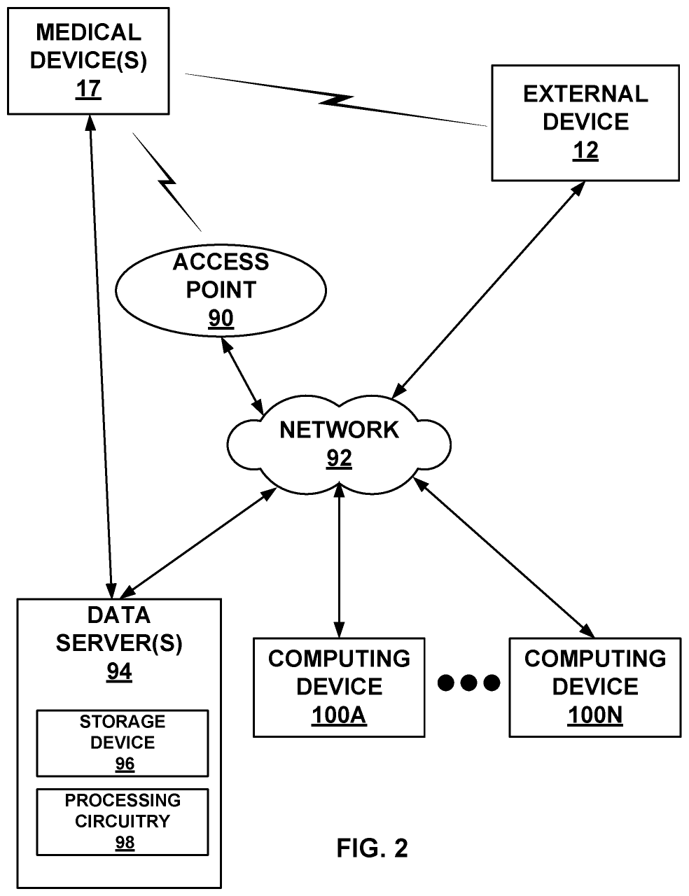

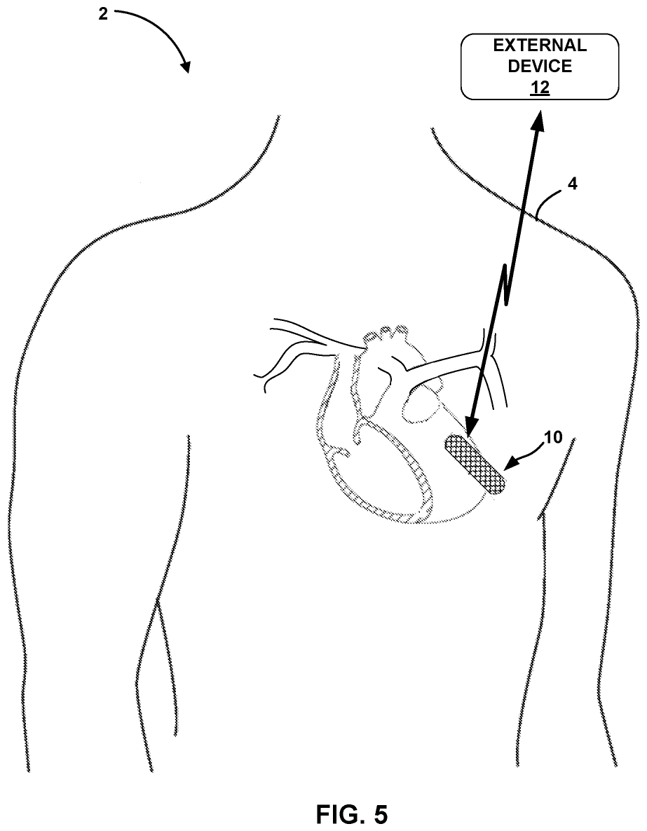

FIG. 2 is a block diagram illustrating an example system that includes one or more medical device(s) 17, an access point 90, a network 92, external computing devices, such as data servers 94, and one or more other computing devices 100A-100N (collectively, “computing devices 100”). In some examples, medical device(s) 17 may include an IMD, such as IMD 10 described with reference to FIGS. 5-7. In this example, medical device(s) 17 may use communication circuitry 54 to communicate with external device 12 via a first wireless connection, and to communicate with an access point 90 via a second wireless connection.

In one or more of the various example techniques described with reference to FIG. 2, access point 90, external device 12, data server(s) 94, and computing devices 100 may be interconnected and may communicate with each other through network 92. Network 92 may include a local area network, wide area network, or global network, such as the Internet. The example system described with reference to FIG. 2 may be implemented, in some aspects, with general network technology and functionality similar to that provided by the Medtronic CareLink® Network, developed by Medtronic, Inc., of Minneapolis, MN.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient.

Medical device(s) 17 may be configured to transmit data, such as sensed, measured, and/or determined values of parameters (e.g., heart rates, impedance measurements, impedance scores, fluid indices, respiratory rate, activity data, cardiac electrograms (EGMs), historical physiological data, blood pressure values, etc.), to access point 90 and/or external device 12. In some examples, medical device(s) 17 may be configured to determine multiple parameters. For example, medical device(s) 17 may include an IMD 10 configured to determine respiration rate values, subcutaneous tissue impedance values, EGM values. In such examples, IMD 10 may provide multiple parameters to serve as evidence nodes to the probability model 19. Access point 90 and/or external device 12 may then communicate the retrieved data to data server(s) 94 via network 92.

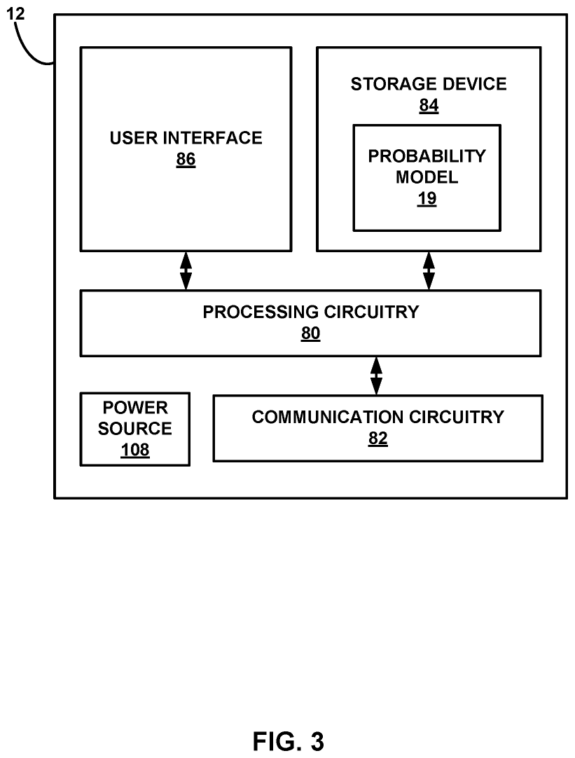

In some instances, one or more of medical device(s) 17 may transmit data over a wired or wireless connection to data server(s) 94 or to external device 12. For example, data server(s) 94 may receive data from medical device(s) 17 or from external device 12. In another example, external device 12 may receive data from data server(s) 94 or from medical device(s) 17, such as parameter values, diagnostic states, or probability scores, via network 92. In such examples, external device 12 may determine the data received from data server(s) 94 or from medical device(s) 17 and may store the data to storage device 84 (FIG. 3) accordingly.

In addition, one or more of medical device(s) 17 may serve as or include data server(s) 94. For example, medical device(s) 17 may include enough storage capacity or processing power to perform the techniques disclosed herein on a single one of medical device(s) 17 or on a network of medical device(s) 17 coordinating tasks via network 92 (e.g., over a private or closed network). In some examples, one of medical device(s) 17 may include at least one of the data server(s) 94. For example, a portable/bedside patient monitor may be able to serve as a data server, as well as serving as one of medical device(s) 17 configured to obtain physiological parameter values from patient 4. In other examples, data server(s) 94 may communicate with each of medical device(s) 17, via a wired or wireless connection, to receive physiological parameter values or diagnostic states from medical device(s) 17. In a non-limiting example, physiological parameter values may be transferred from medical device(s) 17 to data server(s) 94 and/or to external device 12.

In some cases, data server(s) 94 may be configured to provide a secure storage site for data that has been collected from medical device(s) 17 and/or external device 12. In some instances, data server(s) 94 may include a database that stores medical- and health-related data. For example, data server(s) 94 may include a cloud server or other remote server that stores data collected from medical device(s) 17 and/or external device 12. In some cases, data server(s) 94 may assemble data in web pages or other documents for viewing by trained professionals, such as clinicians, via computing devices 100. One or more aspects of the example system described with reference to FIG. 2 may be implemented with general network technology and functionality, which may be similar to that provided by the Medtronic CareLink® Network.

In some examples, one or more of computing devices 100 may be a tablet or other smart device located with a clinician, by which the clinician may program, receive alerts from, and/or interrogate medical device(s) 17. For example, the clinician may access data collected by medical device(s) 17 through a computing device 100, such as when patient 4 is in between clinician visits, to check on a status of a medical condition. In some examples, the clinician may enter instructions for a medical intervention for patient 4 into an application executed by computing device 100, such as based on a status of a patient condition determined by medical device(s) 17, external device 12, data server(s) 94, or any combination thereof, or based on other patient data known to the clinician.

One computing device 100 may transmit instructions for medical intervention to another of computing devices 100 located with patient 4 or a caregiver of patient 4. For example, such instructions for medical intervention may include an instruction to change a drug dosage, timing, or selection, to schedule a visit with the clinician, or to seek medical attention. In further examples, a computing device 100 may generate an alert to patient 4 (or relay an alert determined by a medical device 17, external device 12, or data sever 94) based on a probability score (e.g., posterior probability) determined from parameter values of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 2, data server(s) 94 includes a storage device 96 (e.g., to store data retrieved from medical device(s) 17) and processing circuitry 98. Although not illustrated in FIG. 2 computing devices 100 may similarly include a storage device and processing circuitry. Processing circuitry 98 may include one or more processors that are configured to implement functionality and/or process instructions for execution within data server(s) 94. For example, processing circuitry 98 may be capable of processing instructions stored in memory 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of data server(s) 94 and/or the processing circuitry of computing devices 100 may implement any of the techniques described herein to analyze physiological parameters received from medical device(s) 17, e.g., to determine a probability score of patient 4.

In some examples, storage device 96 of data server(s) 94 may store a probability model 19. In some examples, external device 12 may store probability model 19. For example, data server(s) 94 may transmit probability model 19 to external device 12, where external device 12 may store the probability model 19 in a memory device of external device 12 (not shown in FIG. 2). External device 12 and/or data server(s) 94 may use the probability model 19 to determine a probability score with respect to a health risk for patient 4.

Processing circuitry 98 of data server(s) 94 and/or the processing circuitry of computing devices 100 may also retrieve instructions for utilizing a selected probability model (e.g., one selected using a known selection technique) and execute the probability model to determine the probability score. Processing circuitry 98 of data server(s) 94 and/or the processing circuitry of computing devices 100 may retrieve such data and instructions from storage device 96 or in some instances, from another storage device, such as from one of medical devices 17.

Storage device 96 may include a computer-readable storage medium or computer-readable storage device. In some examples, memory 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

According to one example, processing circuitry may receive, from a medical monitoring device such as medical device(s) 17, a set of one or more patient parameters. The set of one or more patient parameters may, for example include one or more of whether the patient has coronary artery disease, whether the patient has had a prior post myocardial infarction, a heart rate of the patient, a respiration rate of the patient, a respiration volume of the patient, an oxygen saturation level of the patient, a pulse wave transmit time of the patient, a measure of muscle activity of the patient, an ST segment deviation level for the patient, a level of T-wave alternans for the patient, or an activity level of the patient.

The processing circuitry may, for example, be processing circuitry from one or more of external device 12, data servers 94, or computing devices 100. The processing circuitry may determine an exercise tolerance level based on the set of one or more patient parameters and based on at least one comorbidity of the patient. The processing circuitry may also determine the exercise tolerance threshold based on additional parameters that are not acquired from medical device(s) 17. Such parameters may, for example, be inputted by a user and stored in data server(s) 94 of external device 12 and may include indications of comorbidities for the patient. In response to determining that a present activity level for the patient exceeds the exercise tolerance level for the patient, the processing circuitry may send an alert to the patient.

Medical device(s) 17 and/or external device 12 may determine the present activity level for the patient based on a second set of one or more patient parameters. The second set of patient parameter may, for example, include a heart rate of the patient, a respiration rate of the patient, a respiration volume of the patient, a measure of muscle activity of the patient, or an activity level of the patient. In this regard, some of the parameters used to determine the exercise tolerance threshold may also be used to determine the present activity level for the patient. For example, heart rate data collected over a relatively long period of time (e.g., weeks or months) may be used to determine the exercise tolerance threshold, while heart rate data collected over a relatively short period of time (e.g., minutes) may be used to determine the present activity level for the patient.

FIG. 3 is a block diagram illustrating an example configuration of components of external device 12. In some examples, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as one of medical device(s) 17 (e.g., IMD 10). Under the control of processing circuitry 80, communication circuitry 82 may receive downlink telemetry from, as well as send uplink telemetry to, one of medical device(s) 17 (e.g., IMD 10), or another device (e.g., data server(s) 94). Communication circuitry 82 may be configured to transmit or receive signals via inductive coupling, electromagnetic coupling, near-field communication (NFC) technologies (e.g., inductive coupling, NFC or other communication technologies operable at ranges less than 10-20 cm), RF communication, Bluetooth®, Wi-Fi™, or other proprietary or non-proprietary wireless communication schemes. Communication circuitry 82 may also be configured to communicate with devices other than medical device(s) 17 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

Storage device 84 may store one or more probability models 19. Storage device 84 may also store historical data, diagnostic state data, parameter values, probability scores, etc.

Data exchanged between external device 12 and medical device(s) 17 may include operational parameters (e.g., physiological parameter values, diagnostic states, etc.). External device 12 may transmit data including computer readable instructions which, when implemented by medical device(s) 17, may control medical device(s) 17 to change one or more operational parameters and/or export collected data (e.g., physiological parameter values). For example, processing circuitry 80 may transmit an instruction to medical device(s) 17 which requests medical device(s) 17 to export collected data (e.g., heart rate data, blood pressure, ECG records, pulse-transit times, etc.) to external device 12.

In turn, external device 12 may receive the collected data from medical device(s) 17 and store the collected data in storage device 84. Processing circuitry 80 may implement any of the techniques described herein to model parameter values received from medical device(s) 17 to determine diagnostic states, probability scores, etc. Using the modeling techniques disclosed herein, processing circuitry 80 may determine a likelihood that the patient is experiencing an adverse health event (e.g., exercise-induced ischemia or arrhythmia) or is likely to experience an adverse health event within a predetermined amount of time (e.g., within the next 15 minutes, 30 minutes, 60 minutes, etc.). In an illustrative example, the predetermined amount of time may be at least approximately 7 days from when the probability score is determined, such that the probability score indicates the likelihood that an adverse health event will occur in the next 7 days or indicates that the patient is likely already experiencing an adverse health event, such as heart failure decompensation.

External device 12 may be a computing device with a display viewable by a user and an interface for providing input to external device 12 (i.e., a user input mechanism). The user may be a physician technician, surgeon, electrophysiologist, clinician, or patient 4. In some examples, external device 12 may be a notebook computer, tablet computer, computer workstation, one or more servers, cellular phone, personal digital assistant, handheld computing device, networked computing device, or another computing device that may run an application that enables the computing device to interact with IMD 10. External device 12 is configured to communicate with IMD 10 and, optionally, another computing device, via wired or wireless communication. External device 12, for example, may communicate via NFC technologies (e.g., inductive coupling, NFC or other communication technologies operable at ranges less than 10-20 cm) and far-field communication technologies (e.g., Radio Frequency (RF) telemetry according to the 802.11 or Bluetooth® specification sets, or other communication technologies operable at ranges greater than NFC technologies). In some examples, external device 12 may include a programming head that may be placed proximate to the body of patient 4 near the IMD 10 implant site in order to improve the quality or security of communication between IMD 10 and external device 12.

In one example, a user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to medical device(s) 17 (e.g., cardiac EGMs, blood pressure, subcutaneous impedance values, RR, etc.). In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

In some examples, user interface 86 of external device 12 may receive input from the user. The user interface may include, for example, a keypad and a display, which may for example, be a cathode ray tube (CRT) display, an LCD, or an LED display. The keypad may take the form of an alphanumeric keypad or a reduced set of keys associated with particular functions. External device 12 can additionally or alternatively include a peripheral pointing device, such as a mouse, via which the user may interact with the user interface. In some examples, a display of external device 12 may include a touch screen display, and a user may interact with external device 12 via the display. It should be noted that the user may also interact with external device 12 remotely via a networked computing device.

External device 12 may be coupled to external electrodes, or to implanted electrodes via percutaneous leads. In some examples, external device 12 may receive a set of one or more patient parameters from IMD 10. External device 12 may be used to configure operational parameters for IMD 10. For example, external device 12 may provide a parameter resolution for IMD 10 that indicates a resolution of data that IMD 10 should be obtaining. Examples of resolution parameters may include a frequency at which the electrodes process impedance measurements or a frequency at which impedance measurements should be considered in determining a diagnostic state.

Power source 108 delivers operating power to the components of external device 12. Power source 108 may include a battery and a power generation circuit to produce the operating power. In some embodiments, the battery may be rechargeable to allow extended operation. Recharging may be accomplished by electrically coupling power source 108 to a cradle or plug that is connected to an alternating current (AC) outlet. In addition or alternatively, recharging may be accomplished through proximal inductive interaction between an external charger and an inductive charging coil within external device 12. In other embodiments, traditional batteries (e.g., nickel cadmium or lithium ion batteries) may be used. In addition, external device 12 may be directly coupled to an alternating current outlet to power external device 12. Power source 108 may include circuitry to monitor power remaining within a battery. In this manner, user interface 86 may provide a current battery level indicator or low battery level indicator when the battery needs to be replaced or recharged. In some cases, power source 108 may be capable of estimating the remaining time of operation using the current battery.

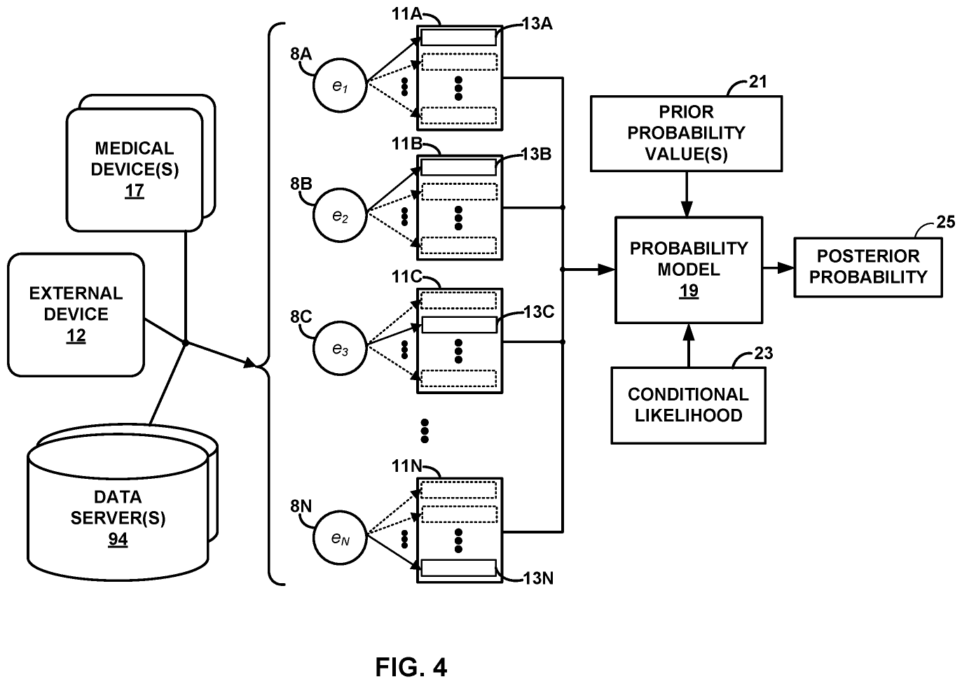

FIG. 4 illustrates a framework that uses a medical system, such as system 2, to determine a patient-specific exercise tolerance threshold for patient 4. The medical system may include external device 12 or one or more of data server(s) 94. Although primarily described in terms of one or more data server(s) 94 determining the exercise tolerance threshold, it will be understood that any one or more devices (e.g., processing circuitry of such devices), such as external device 12, one or more medical devices 17, or computing devices 100, may perform the exercise tolerance threshold determination using probability model 19 as described herein. In any event, FIG. 4 illustrates external device 12, medical device(s) 17, and/or data server(s) 94 as being configured to supply input to probability model 19.

In some examples, storage device 96 of data server(s) 94 may store the parameter values that relate to one or more parameters, which may have been received from one or more other devices of the system via network 92. Data server(s) 94 may store the parameter values as raw data or as conditioned data via signal processing techniques. For example, data server(s) 94 may store, within storage device 96, changes in PTT, which may be proportional to a change in blood pressure, occurrences of arrhythmias (premature ventricular contractions (PVCs), supraventricular tachycardia (SVT)), changes in oxygen saturation, or ST deviation values as values determined from ECG data collected by one or more medical device(s) 17. In some examples, processing circuitry 98 of data server(s) determines ST deviation values based on ECG data measured by one or more medical device(s) 17. In some examples, processing circuitry of the medical device(s) or processing circuitry 80 of external device 12 determines the ST deviation values.

Processing circuitry, e.g., processing circuitry 98 of data server(s) 94, may store data received from medical device(s) 17 (e.g., from IMD 10) to a storage device, e.g., storage device 96 of data server(s) 94. In an example, storage device 96 may be configured to store measured and/or determined values of any of the various parameters described herein. In some examples, data server 94 may receive parameter value measurements (e.g., raw or conditioned data) from medical device(s) 17, e.g., IMD 10, via network 92. In such examples, processing circuitry 98 of data server(s) 94 may determine the index or score values used to determine inputs to probability model 19. In some examples, medical device(s) 17, e.g., IMD 10, or external device 12 or another device may determine the index or score values from one or more parameter values. Data server(s) 94 may receive data from medical device(s) 17 and determine, via probability model 19, an exercise tolerance threshold based on the data.

With reference still to FIG. 4, processing circuitry 98 of data server(s) (or processing circuitry of any other device of the system) may, in some examples, perform the probability score determination using probability model 19 in accordance with the following. As described herein, processing circuitry 98 may be coupled to one or more storage devices such that processing circuitry 98 may leverage the various data repositories in order to determine the probability score.

In some examples, processing circuitry 98 may be configured to determine a respective one or more values for each of a plurality of parameters. For instance, processing circuitry may determine one or more values for a first parameter and one or more values for a second parameter. In one example, the values may correspond to measurement readings determined via medical device(s) 17 or other information indicative of a health status of patient 4. For example, the values may include respiration rate values, ECG values, activity level values, etc. For example, the values may indicate at when patient 4 was active or when patient 4 was inactive. The values may include accelerometer values that indicate a posture of patient 4 or a change in the posture of patient 4 over time (e.g., a posture-change count). Posture change count may be based on z-axis accelerometer values. Other values may include periodic x, y, and z-axis accelerometer measurements. The values may also indicate the presence of comorbidities for patient 4, such as a prior arrythmia, CAD, prior MI, hypertension, diabetes, COPD, or obesity.

In some examples, processing circuitry 98 may adjust the criteria for determining each diagnostic state as more information becomes available to processing circuitry 98 over time. For example, processing circuitry 98 may determine that one or more diagnostic states for one or more parameters may be optimized. In some examples, processing circuitry 98 may determine such optimization potential based on the output of a ML model trained on posterior probability data, diagnostic states, and criteria performance data. In any event, processing circuitry 98 may adjust the criteria used for determining the one or more diagnostic states for one or more parameters.

In some examples, processing circuitry 98 may be configured to identify diagnostic states 11A-11N (collectively, “diagnostic states 11”) for each of the parameters based on the respective values. For example, various thresholds may be used to determine a diagnostic state of a parameter. In some examples, processing circuitry 80 is configured to select, from at least three potential diagnostic states, a single diagnostic state for each of evidence nodes 8. For example, the diagnostic state may be high, medium, low, where some parameters may include more or less diagnostic states (H and L). In the example described with reference to FIG. 4, the diagnostic states are selected from N number of diagnostic states. In a few examples, diagnostic state 13A has a first diagnostic state (e.g., high or very high), where diagnostic state 13C has a second diagnostic state, where the first diagnostic state may include a high risk categorization and the second diagnostic state may include a medium risk categorization depending on the respective one or more parameter values. Further, diagnostic state 13B corresponding to evidence node 8B may be high or very high and diagnostic state 13N corresponding to evidence node 8N may be low or very low.

The diagnostic states may be determined independently for each parameter. For example, processing circuitry 98 may compare the values obtained for a first parameter to one or more thresholds to determine a diagnostic state for the first parameter independently of processing circuitry comparing values obtained for a second parameter to one or more thresholds to determine a diagnostic state for the second parameter. In some examples, data server(s) 94 may receive the diagnostic state for one or more of the diagnostic states 11. In other examples, data server(s) 94 may determine the diagnostic state for the diagnostic state for one or more of the diagnostic states 11 based on the respective values of the parameters. In any event, diagnostic states 11 define evidence nodes 8 for probability model 19. In other words, diagnostic states 11 serve as evidence nodes 8 for probability model 19.

In some examples, probability model 19 may include a Bayesian framework or BBN. Other suitable probability models may be used to determine probability scores given diagnostic states of parameters. For example, a Bayesian ML model may be used to determine probability scores based on diagnostic states of parameters. Processing circuitry may train probability model 19 on values associated with the parameters. In other examples, processing circuitry may include as input to probability model 19 prior probability value(s) 21 or conditional likelihood 23. For example, processing circuitry 98 may determine, from the plurality of parameters, prior probability value 21. The prior probability value 21 may be determined from existing data. Processing circuitry 98 may also determine, from the plurality of parameters, conditional likelihood parameter 23.

In some examples, processing circuitry 98 may determine the conditional likelihood, or P(eᵢ|d), from existing data. Processing circuitry 98 may utilize existing data from one or more patients or subjects, where the existing data is then used to determine conditional likelihood parameters of a model utilizing a probability theorem, such as Bayes rule.

In one example, the value of ‘d’ may represent the presence or absence of an (exercise-induced) cardiac event or other adverse health event. As such, processing circuitry 98 may use earlier data to determine whether a particular diagnostic criterion was satisfied before a cardiac event (e.g., P(eᵢ|d=1)) or whether the particular diagnostic criterion was satisfied when there was no cardiac event (e.g., P(eᵢ|d=0)). In an example, using a first evidence node e₁ as corresponding to a specific comorbidity, processing circuitry 98 may determine from a plurality of existing data points the conditional likelihood for: P(e₁=H|d=1), P(e₁=H|d=0), P(e₁=M|d=1), P(e₁=M|d=0), P(e₁=L|d=1), and P(e₁=L|d=0). That is, processing circuitry 98 may determine the conditional likelihood from data derived from True Positives, False Positives, False Negatives, and False Positives. In some examples, processing circuitry 98 use the same data to provide a desired sensitivity and specificity of HF detection. That is, posterior probability 25 may represent an estimate of positive predictive value (PPV) based on sensitivity, specificity and event rate (e.g., prior probability 21).

Processing circuitry 98 may determine the conditional likelihoods for each parameter used as an input evidence node to probability model 19 (e.g., each of eᵢ). In some examples, processing circuitry 98 may then utilize the conditional likelihood probabilities to determine the probability model 19. In such examples, the determined probability model 19 may include a computable joint distribution.

As such, processing circuitry 98 may identify prior probability value 21 and/or the conditional likelihood parameter 23 as inputs to probability model 19 when determining the probability score. In such examples, the probability model may be expressed as:

$$P ( d , {e}_{1} , … , {e}_{N} ) = P ( d ) {\prod }_{i = 1}^{N} P ( {e}_{i} \vert d ) ,$$

wherein P(d) represents the prior probability value, P(eᵢ|d) represents the conditional likelihood parameter, d represents a parent node, and e₁-e_{N} represent the evidence nodes.

In some examples, processing circuitry 98 may be configured to determine a probability score from probability model 19 based on evidence nodes 8. Processing circuitry 98 may then determine a patient-specific exercise threshold for a patient based on the probability score. In an example, the BBN may have one or more child nodes (e.g., n-nodes as shown in FIG. 4) and a parent node, represented by posterior probability 25.

The probability score may include a likelihood that the patient is experiencing an adverse health event or is likely to experience the adverse health event within a predetermined amount of time at a certain activity level. In an example, the adverse health event could be a worsening cardiac event (e.g., HF decompensation). The probability score may be expressed in terms of a percentage, a decimal number, or a threshold categorization, such as 50%, 0.5, or medium likelihood, where in this example, 50% corresponds to a threshold categorization of medium likelihood. In some examples, the probability score may be expressed in terms of a range such as >50% or between 50-60%. For example, the probability score may indicate that patient 4 has a 50% chance of experiencing an adverse health event at a certain activity level.

In some examples, processing circuitry, e.g., processing circuitry 50 of IMD 10, processing circuitry 80 of external device 12, or processing circuitry 98 of data server(s) 94, may determine the probability score on a daily basis. For example, processing circuitry 98 may determine the probability score every day based on data corresponding to a previous X number of days. In some examples, processing circuitry 98 may store in storage device 96 diagnostic states for various parameters each day for a finite number of days, such as in a first in, first out (FIFO) buffer or sliding window. In some examples, processing circuitry 98 may store the last 30 diagnostic states for each parameter determined on a daily basis for the past 30 days. For example, processing circuitry 98 may store the last 30 diagnostic states for impedance scores determined on a daily basis for the past 30 days, store the last 30 diagnostic states for RR determined on a daily basis for the past 30 days, etc. Processing circuitry 98 may determine the probability score at a predefined time interval each day using the previous 30 diagnostic states of each parameter determined over the past 30 days as input to the probability model 19. In another example, processing circuitry 98 may determine the probability score at a predefined time interval each day using the previous 15 diagnostic states of each parameter determined over the past 15 days as input to the probability model 19. In any event, processing circuitry 98 may receive data from medical device(s) 17 on a periodic basis, such as on a daily, weekly, or biweekly basis, etc. In such examples, processing circuitry 98 may determine the probability score upon receiving the data from medical device(s) 17 according to the periodic transmission rate of medical device(s) 17 (e.g., daily, weekly, biweekly, etc.). That is, in one example, processing circuitry 98 may determine diagnostic states (e.g., risk states) for each parameter. In such examples, processing circuitry 98 may combine the last X number of days of diagnostic states together to determine a probability score using probability model 19.

In another example, processing circuitry 98 may determine the probability score (and a corresponding exercise tolerance threshold) and diagnostic states on a periodic basis. In addition, processing circuitry 98 may determine the status of the health condition of patient 4 using the probability score and a threshold on a periodic basis. In a non-limiting example, processing circuitry 98 may compute the probability score, diagnostic states, and status on a daily basis. In such examples, processing circuitry 98 may store the probability score and/or diagnostic state for the last X number of days, such as for the last 30 days. In some examples, processing circuitry 98 may determine the probability score on a day basis using diagnostic data from the past X number of days, such as the last 30 days. In such examples, processing circuitry 98 may determine, on any given day, that the probability score satisfies a threshold. For example, processing circuitry 98 may determine that the probability score exceeds a threshold. In such examples where processing circuitry 98 determines that probability score satisfies a threshold, processing circuitry 98 may transmit an alert externally, such as to a physician device or patient device.

Although described with reference to processing circuitry 98, the techniques of this disclosure are not so limited. In some examples, other processing circuitry (e.g., processing circuitry 80, processing circuitry 50, or processing circuitry of another one of medical device(s) 17, such as a CPU of one of medical device(s) 17) may perform one or more of the techniques of this disclosure and may coordinate with other devices accordingly. For example, processing circuitry of one of medical device(s) 17 may determine the probability score on a daily basis, compare the probability score to a threshold, and cause the transmission of an alert where the probability score satisfies the threshold. In such examples, a particular medical device from one of medical device(s) 17 may receive data (e.g., diagnostic data) from network 92, such as from other medical device(s) 17, external device 12, or data server(s) 94, and may determine the probability score using processing circuitry included with the particular medical device.

In examples where storage device 96 does not have data corresponding to a particular timeframe, processing circuitry 98 may extrapolate data or interpolate data or in some examples, processing circuitry 98 may determine an extent to which the data for a parameter is missing and determine whether to use the parameter when determining the probability score, as discussed herein.

In another example, processing circuitry 98 may determine the probability score based on determined values of the parameters that correspond to a preceding timeframe relative to when the probability score is determined. For example, processing circuitry 98 may identify diagnostic states based on parameter values determined during a preceding timeframe prior to determining the probability score. In one example, the preceding timeframe may be approximately 30 days relative to when the probability score is determined. In some examples, the preceding timeframe and the predetermined amount of time may include the same amount of time relative to when the probability score is determined. For example, where the predetermined amount of time is 30 days in the future, the preceding timeframe may be 30 days of past data, and where the predetermined amount of time is 29 days in the future, the preceding timeframe may be 29 days in the past.

In some examples, the amount of time for each of the predetermined amount of time and the preceding timeframe may be different based on the number of days in each month. For example, processing circuitry 98 may determine a probability score on the last day of each month and for convenience, may forecast for the next month based on the parameters determined for the preceding month, in which case the predetermined amount of time and the preceding timeframe may include a different amount of time. In other examples, the amount of time for each may remain constant regardless of convenience factors (e.g., 60 days on either end, or 30 days for the predetermined amount of time in the future and 45 days for the preceding timeframe).

In some examples, the amount of time for the preceding timeframe may be dependent on values associated with the parameters. For example, processing circuitry 98 may determine diagnostic states for subcutaneous tissue impedance scores on a 30-day preceding timeframe basis, whereas processing circuitry 98 may determine diagnostic states for respiration rate on a shorter or longer preceding timeframe basis. In such examples, processing circuitry 98 may use a plurality of preceding timeframes for various parameters. In other examples, processing circuitry 98 may use a common preceding timeframe regardless of any resolution parameters used to determine the parameter values, where resolution parameters may include filters, time constraints, or activity determinations.

With reference still to FIG. 4, processing circuitry 98 is configured to identify, from the respective one or more values for each parameter, a plurality of parameter features that encode amplitude, out-of-normal range values, and temporal changes. In the example of amplitude, a physiological parameter feature may encode R-wave amplitudes, accelerometer signal amplitudes, etc. For example, processing circuitry 98 may determine whether a particular parameter satisfies an absolute threshold. In an illustrative example, processing circuitry 98 may determine whether an average NHR of patient 4 is greater than a predefined threshold of 90 bpm. In the example of out-of-range values, a parameter feature may encode use range values to determine whether a parameter includes out-of-range values to encode. For example, processing circuitry 98 may determine a high heart rate based on expected heart rate values.

In an illustrative example, processing circuitry 98 may determine NHR out-of-range values by comparing the average NHR to determine how many NHR values have been greater than 90 bpm or less than 55 bpm. In the example of temporal changes, a parameter feature may encode changes in a parameter over time. In one example, processing circuitry 98 may encode a feature of subcutaneous impedance measurements with changes in impedance over a period of days or weeks. Similar to calculating the fluid index using impedance values, processing circuitry 98 may determine relative changes in a parameter value to determine temporal changes, rather than absolute changes. In an illustrative example, processing circuitry 98 may determine whether an average or current-day NHR value has increased in a sustained manner over the last 7 days or 30 days relative to NHR values in the last 7 days or 30 days.

In such examples, processing circuitry 98 is configured to identify the evidence nodes based at least in part on the plurality of parameter features. For example, processing circuitry 98 may extract features that encode information regarding out-of-normal range values, as well as temporal changes at weekly and monthly time scale for the parameters. In a non-limiting example, processing circuitry 98 may determine diagnostic categories based on a combination of features. For example, the category with the largest number of HFH rates may be designed to have the lowest occurrence rate through the feature extraction process.

In some examples, processing circuitry 98 is configured to identify a plurality of parameter features based on the respective one or more values for each parameter. The parameter features are configured to, upon analysis, yield a same number of potential diagnostic states for each parameter. In some examples, the same number of potential diagnostic states may be three potential diagnostic states (e.g., H, M, and L). In other examples, one or more parameters may have a different number of potential diagnostic states, such as one or two potential diagnostic states. For example, AF may have two diagnostic states of high and low. In such examples, processing circuitry 98 is configured to identify, from the potential diagnostic states, the diagnostic state for each of the parameters.

Processing circuitry 98 may extract features from the parameters and/or from the parameter values. For example, processing circuitry 98 may analyze a large set of time series data for each parameter for time windows including the number of days the values are outside a normal amplitude range, cumulative sum of difference between the raw measurement and an adaptive reference (CSAR), cumulative sum of difference between the raw measurement in a fixed reference (CSFR), number of days CSAR or CSFR were above a threshold, slope or rate of change of raw measurement values, or mean, median, minimum, and maximum measurement values. Processing circuitry 80 may extract such features for each parameter to encode amplitude and temporal characteristics with respect to particular temporal scales.

In some examples, processing circuitry 98 may determine a MVN as one of the evidence nodes. For instance, multiple parameters may factor into determining a single child node of evidence nodes 8. In a non-limiting example, processing circuitry 98 is configured to determine an input to a first child node of evidence nodes 8 based on a combination of one or more values. For example, one evidence node may be based on a combination of an indication of atrial fibrillation (AF) extent in patient 4 during a time period and one or more values indicating a ventricular rate during the time period (e.g., during AF). In addition, processing circuitry 98 may be configured to determine an input to a second child node of the plurality of evidence nodes based on the respective one or more values of the one or more subcutaneous tissue impedance parameters. In such instances, evidence node 8A may include a combination of an AF extent indication value(s) and ventricular rate value(s), whereas evidence node 8A may indicate one or more other parameter values.

With reference still to FIG. 4, processing circuitry 98 may determine, for each of the plurality of parameters or evidence nodes 8, the respective one or more parameter values at various frequencies. For example, processing circuitry 98 may determine the values for evidence node 8A at a different frequency than for evidence node 8B. Thus, diagnostic states 11 may update at different frequencies. In such examples, processing circuitry 98 may delay execution of probability model 19 until an appropriate number of diagnostic states are deemed current or updated. In any event, processing circuitry 80 may determine the diagnostic states using the respective one or more values. Processing circuitry 98 may use the diagnostic states to determine posterior probability 25. Processing circuitry 98 may then store, the respective one or more values and/or the probability score to, as examples, storage device 96, storage device 84, and/or storage device 56 of medical device(s) 17 (e.g., IMD 10).

FIG. 5 illustrates the environment of an example medical system 2 in conjunction with a patient 4, in accordance with one or more techniques of this disclosure. Patient 4 ordinarily, but not necessarily, will be a human. For example, patient 4 may be an animal needing ongoing monitoring for cardiac conditions.

In some examples, system 2 may include IMD 10. In other examples, system 2 may not include IMD 10 and may instead include other medical device(s) 17 (not shown in FIG. 5). IMD 10 may include one or more electrodes (not shown) on its housing, or may be coupled to one or more leads that carry one or more electrodes. System 2 may also include external device 12 and, although not depicted in FIG. 5, the various other devices illustrated in one or more of the various example techniques described with reference to FIG. 2. Example system 2 may be used to measure subcutaneous impedance to provide to patient 4 other users an early warning for the onset of a heart failure decompensation event.

The example techniques may be used with an IMD 10, which may be in wireless communication with at least one of external device 12 or data server(s) 94. In some examples, IMD 10 is implanted outside of a thoracic cavity of patient 4 (e.g., subcutaneously in the pectoral location illustrated in FIG. 5). IMD 10 may be positioned near the sternum near or just below the level of the heart of patient 4, e.g., at least partially within the cardiac silhouette. IMD 10 may include a plurality of electrodes and may be configured for subcutaneous implantation outside of a thorax of patient 4.

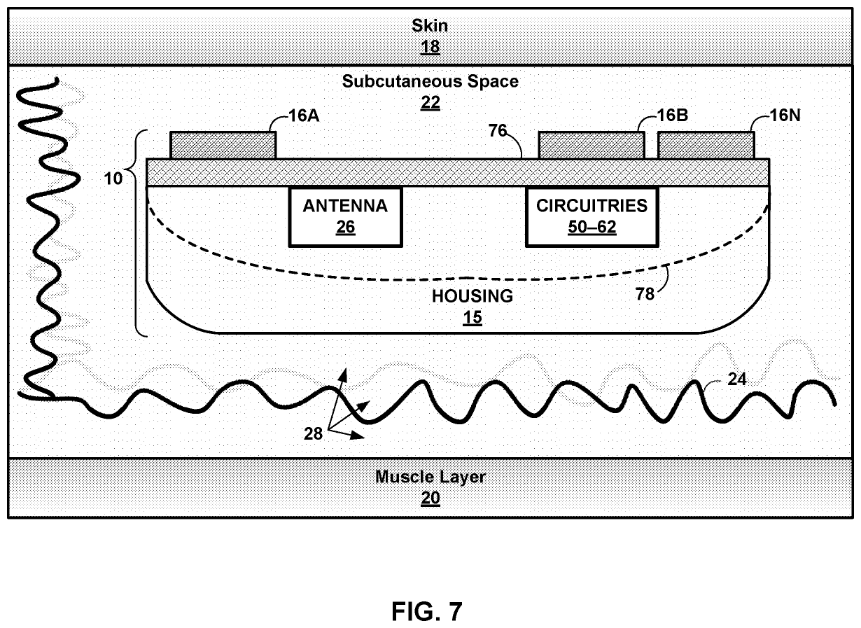

Accordingly, impedance measurements taken via electrodes in the subcutaneous space, e.g., electrodes on a subcutaneously implanted medical device as shown in FIGS. 7-9, may be measurements of the impedance of interstitial fluid and subcutaneous tissue. In an example, during a heart failure decompensation event, reduction in cardiac output can tend to increase venous pressure. An increase in venous pressure tends to lead to an increase in pressure with respect to capillaries compared to the interstitial space. The combination of such tendencies may then lead to a net outflow of fluid from the capillaries into the interstitium or interstitial space of a patient. In such instances, the interstitium will have an increase in fluid accumulation. An increase in fluid accumulation tends to provide a reduction in impedance measured between electrodes.

Implantable medical devices (IMDs) can sense and monitor impedance signals and use those signals to determine a health condition status of a patient or other health condition status of a patient (e.g., edema, preeclampsia, hypertension, etc.). The electrodes used by IMDs to sense impedance signals are typically integrated with a housing of the IMD and/or coupled to the IMD via one or more elongated leads. Example IMDs that include electrodes include the Reveal LINQ™ or LINQ II™ Insertable Cardiac Monitor (ICM), developed by Medtronic, Inc., of Minneapolis, MN, which may be inserted subcutaneously. Other example IMDs may include electrodes on a subcutaneous lead connected to another one of medical device(s) 17, such as a subcutaneous implantable cardioverter-defibrillator (ICD) or an extravascular ICD. Such IMDs may facilitate relatively longer-term monitoring of patients during normal daily activities and may periodically transmit collected data to a network service, such as the Medtronic CareLink® Network.

Medical devices configured to measure impedance via implanted electrodes, including the examples identified herein, may implement the techniques of this disclosure for measuring impedance changes in the interstitial fluid of a patient to determine whether the patient is experiencing worsening heart failure or decompensation. The techniques include evaluation of the impedance values using criteria configured to provide a desired sensitivity and specificity of heart failure detection. The techniques of this disclosure for identifying heart failure worsening may facilitate determinations of cardiac wellness and risk of sudden cardiac death and may lead to clinical interventions to suppress heart failure worsening, such as with medications.

As such, IMD 10 may be configured to measure, in some cases among other physiological parameter values, impedance values within the interstitial fluid of patient 4. For example, IMD 10 may be configured to receive one or more signals indicative of subcutaneous tissue impedance from electrodes 16. In some examples, IMD 10 may be a purely diagnostic device. For example, IMD 10 may be a device that only determines subcutaneous impedance parameters of patient 4, or a device that determines subcutaneous impedance parameters as well as other physiological parameter values of patient 4. IMD 10 may use the impedance value measurements to determine one or more fluid index values, impedance scores, and/or various thresholds, such as adaptive thresholds, scoring thresholds, weighting factors for thresholds, and/or cardiac risk thresholds.

System 2 may be configured to measure subcutaneous impedance of patient 4 and processes impedance data to accumulate evidence of a respiration rate of patient 4. In some examples, IMD 10 may also sense cardiac electrogram (EGM) signals via the plurality of electrodes and/or operate as a therapy delivery device. For example, IMD 10 may additionally operate as a therapy delivery device to deliver electrical signals to the heart of patient 4, such as an implantable pacemaker, a cardioverter, and/or defibrillator, a drug delivery device that delivers therapeutic substances to patient 4 via one or more catheters, or as a combination therapy device that delivers both electrical signals and therapeutic substances.

In some examples, system 2 may include any suitable number of leads coupled to IMD 10, and each of the leads may extend to any location within or proximate to a heart or in the chest of patient 4. For example, other examples therapy systems may include three transvenous leads and an additional lead located within or proximate to a left atrium of a heart. As other examples, a therapy system may include a single lead that extends from IMD 10 into a right atrium or right ventricle, or two leads that extend into a respective one of a right ventricle and a right atrium.

In some examples, IMD 10 may be implanted subcutaneously in patient 4. Furthermore, in some examples, external device 12 may monitor subcutaneous impedance values. In some examples, IMD 10 takes the form of the Reveal LINQ™ ICM, or another ICM similar to, e.g., a version or modification of, the LINQ™ ICM, which may be inserted subcutaneously. Such IMDs may facilitate relatively longer-term monitoring of patients during normal daily activities, and may periodically transmit collected data to a network service, such as the Medtronic CareLink® Network.

Although described in the context of examples in which IMD 10 includes an insertable or implantable IMD, example systems including one or more external devices of any type configured to sense subcutaneous tissue impedances may be configured to implement the techniques of this disclosure. In some examples, IMD 10 may be a device configured to measure impedances of a fluid and shifts in impedances of the fluid, such as interstitial fluid. For example, IMD 10 may have one or more electrodes disposed within one layer of patient 4 (e.g., subcutaneous layer), whereas at least one other electrode may be disposed within another layer of patient 4 (e.g., dermis layer, muscle layer, etc.). In such examples, IMD 10 may be able to measure impedances and shifts in impedances of the interstitial fluid of the subcutaneous layer. In another example, IMD 10 may be a cutaneous patch device having electrodes on the outside of the skin. In such examples, IMD 10 may use the cutaneous patch device to measure impedances and shifts in impedances of the interstitial fluid in the subcutaneous layer.

In examples in which IMD 10 also operates as a pacemaker, a cardioverter, and/or defibrillator, or otherwise monitors the electrical activity of the heart, IMD 10 may sense electrical signals attendant to the depolarization and repolarization of the heart of patient 4 via electrodes of or coupled to IMD 10, e.g., which may include the electrodes used to determine subcutaneous impedance. In some examples, IMD 10 can provide pacing pulses to the heart of patient 4 based on the electrical signals sensed within the heart of patient 4. The configurations of electrodes used by IMD 10 for sensing and pacing may be unipolar or bipolar. IMD 10 may also provide defibrillation therapy and/or cardioversion therapy via electrodes located on at least one lead, as well as a housing electrode. IMD 10 may detect tachyarrhythmia of the heart of patient 4, such as fibrillation of atria or ventricles, and deliver defibrillation or other tachyarrhythmia therapy to the heart of patient 4 in the form of electrical pulses. In some examples, IMD 10 may be programmed to deliver a progression of therapies, e.g., pulses with increasing energy levels, until a fibrillation of the heart of patient 4 is stopped. IMD 10 detects fibrillation or other tachyarrhythmias employing tachyarrhythmia detection techniques known in the art.

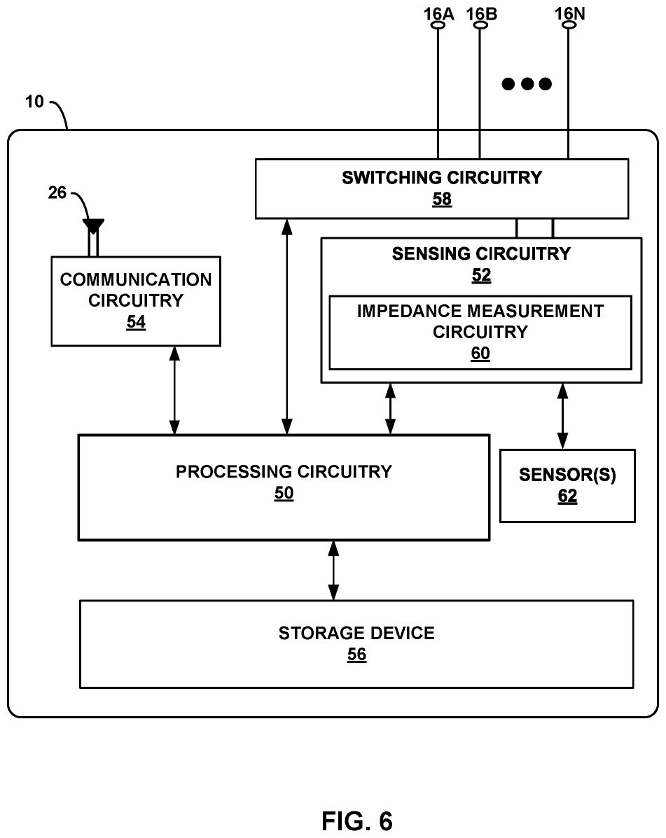

FIG. 6 is a functional block diagram illustrating an example configuration of IMD 10. IMD 10 may include an example of one of medical device(s) 17 described with reference to FIGS. 2-4. In the illustrated example, IMD 10 includes electrodes 16A-16N (collectively, “electrodes 16”), antenna 26, processing circuitry 50, sensing circuitry 52, impedance measurement circuitry 60, communication circuitry 54, storage device 56, switching circuitry 58, sensors 62. IMD 10, along with other medical device(s) 17, may also include a power source. In general, the power source may include a rechargeable or non-rechargeable battery. Each of medical device(s) 17 may include components common to those of IMD 10. For example, each of medical device(s) 17 may include processing circuitry 50. For sake of brevity, each configuration of each medical device(s) 17 will not be described in this application. That is, certain components of IMD 10 may serve as representative components of other medical device(s) 17 (e.g., storage device 56, communication circuitry 54, sensor(s) 62, etc.).

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16 via switching circuitry 58, e.g., to select the electrodes 16 and polarity, referred to as the sensing vector, used to sense impedance and/or cardiac signals, as controlled by processing circuitry 50. Sensing circuitry 52 may sense signals from electrodes 16, e.g., to produce a cardiac EGM, in order to facilitate monitoring the electrical activity of the heart. Sensing circuitry 52 also may monitor signals from sensors 62, which may include one or more accelerometers, pressure sensors, and/or optical sensors, as examples. In some examples, sensing circuitry 52 may include one or more filters and amplifiers for filtering and amplifying signals received from electrodes 16 and/or sensors 62.

In some examples, processing circuitry 50 may use switching circuitry 58 to select, e.g., via a data/address bus, which of the available electrodes are to be used to obtain impedance measurements of interstitial fluid and to sense cardiac signals, and to select the polarities of the electrodes. Switching circuitry 58 may include a switch array, switch matrix, multiplexer, transistor array, microelectromechanical switches, or any other type of switching device suitable to selectively couple sensing circuitry 58 to selected electrodes. In some examples, sensing circuitry 52 includes one or more sensing channels, each of which may include an amplifier. In response to the signals from processing circuitry 50, switching circuitry 58 may couple the outputs from the selected electrodes to one of the sensing channels.

In some examples, one or more channels of sensing circuitry 52 may include one or more R-wave amplifiers that receive signals from electrodes 16. In some examples, the R-wave amplifiers may take the form of an automatic gain-controlled amplifier that provides an adjustable sensing threshold as a function of the measured R-wave amplitude. In addition, in some examples, one or more channels of sensing circuitry 52 may include a P-wave amplifier that receives signals from electrodes 16. Sensing circuitry may use the received signals for pacing and sensing in the heart of patient 4. In some examples, the P-wave amplifier may take the form of an automatic gain-controlled amplifier that provides an adjustable sensing threshold as a function of the measured P-wave amplitude. Other amplifiers may also be used.

In some examples, processing circuitry 50 may be configured to record an R-wave amplitude for an ECG sensed by sensing circuitry 52. For example, sensing circuitry 52 may be configured to sense a subcutaneous ECG, and processing circuitry 50 may be configured to record an R-wave amplitude of the subcutaneous ECG. In another example, sensing circuitry 52 may be configured to record cardiac electrogram using leads in the heart of patient 4 and as measured between the housing of one of medical device(s) 17 (e.g., a can) and the leads in the heart of patient 4, and processing circuitry 50 may be configured to record an R-wave amplitude of the cardiac electrogram. Similarly, sensing processing circuitry 50 may record a R-wave slopes or R-wave widths for an ECG or other cardiac electrogram.

In some examples, sensing circuitry 52 includes a channel that includes an amplifier with a relatively wider pass band than the R-wave or P-wave amplifiers. Signals from the selected sensing electrodes that are selected for coupling to this wide-band amplifier may be provided to a multiplexer, and thereafter converted to multi-bit digital signals by an analog-to-digital converter for storage in storage device 56. In some examples, processing circuitry 50 may employ digital signal analysis techniques to characterize the digitized signals stored in storage device 56 to detect P-waves (e.g., within ventricular or far-field signals and instead of or in addition to use of P-wave amplifiers) and classify cardiac tachyarrhythmias from the digitized electrical signals.

Based on the detection R-waves and P-waves, e.g., their rates, processing circuitry 50 may identify atrial and ventricular tachyarrhythmias, such as AF or VF. Processing circuitry may employ digital signal analysis techniques to detect or confirm such tachyarrhythmias in some examples. Processing circuitry 50 may determine values of parameters based on detection of such tachyarrhythmias, and a probability of a health event may be determined based on the parameter values according to the techniques described herein. Example parameters determined based on detection of tachyarrhythmia include an extent, e.g., frequency and/or duration during a time period, of AF or other tachyarrhythmias.

Processing circuitry 50 may also determine other parameter values that can be used to determine probability of a health event based on the cardiac EGM and detection of depolarizations therein. As examples, processing circuitry 50 may determine one or more heart rate values, such as night heart rate values, one or more heart rate variability values. As other examples, processing circuitry 50 may determine magnitudes of or intervals between features within the cardiac EGM, such as depolarization amplitudes, depolarization widths, or intervals between depolarizations and repolarizations.

In some examples, sensors 62 include one or more accelerometers or other sensors configured to generate signals that indicate motion and orientation of patient 4, e.g., that indicate activity level or posture of the patient. In some examples, processing circuitry 50 processes such signals to determine values of one or more parameters that can be used to determine probability of a health event. For example, processing circuitry 50 may quantify duration, frequency, and/or intensity of activity and/or posture changes, e.g., daily or during some other period. In some examples, processing circuitry 50 may determine an amount of time patient spends inactive, e.g., sleeping, but not in a supine posture based on such signals.

Sensing circuitry 52 includes impedance measurement circuitry 60. Processing circuitry 50 may control impedance circuitry 60 to periodically measure an electrical parameter to determine an impedance, such as a subcutaneous impedance indicative of fluid found in interstitium 28. For a subcutaneous impedance measurement, processing circuitry 50 may control impedance measurement circuitry 60 to deliver an electrical signal between selected electrodes 16 and measure a current or voltage amplitude of the signal. Processing circuitry 50 may select any combination of electrodes 16, e.g., by using switching circuitry 58 and sensing circuitry 52. Impedance measurement circuitry 60 includes sample and hold circuitry or other suitable circuitry for measuring resulting current and/or voltage amplitudes. Processing circuitry 50 determines an impedance value from the amplitude value(s) received from impedance measurement circuitry 60.

Because either IMD 10 or external device 12 may be configured to include sensing circuitry 52, impedance measurement circuitry 60 may be implemented in one or more processors, such as processing circuitry 50 of IMD 10 or processing circuitry 80 of external device 12. Impedance measurement circuitry 60 is, in this example, shown in conjunction with sensing circuitry 52 of IMD 10. Impedance measurement circuitry 60 may be embodied as one or more hardware modules, software modules, firmware modules, or any combination thereof. Impedance measurement circuitry 60 may analyze impedance measurement data on a periodic basis to identify a decrease in subcutaneous impedance in patient 4 and alert patient 4 when the decrease indicates onset of a possible heart failure decompensation event.

In some examples, processing circuitry 50 may perform an impedance measurement by causing impedance measurement circuitry 60 (via switching circuitry 58) to deliver a voltage pulse between at least two electrodes 16 and examining resulting current amplitude value measured by impedance measurement circuitry 60. In some examples, switching circuitry 58 delivers signals that do deliver stimulation therapy to the heart of patient 4. In other examples, these signals may be delivered during a refractory period, in which case they may not stimulate the heart of patient 4.

In some examples, processing circuitry 50 may perform an impedance measurement by causing impedance measurement circuitry 60 (via switching circuitry 58) to deliver a current pulse across at least two selected electrodes 16. Impedance measurement circuitry 60 holds a measured voltage amplitude value. Processing circuitry 50 determines an impedance value based upon the amplitude of the current pulse and the amplitude of the resulting voltage that is measured by impedance measurement circuitry 60. IMD 10 may use defined or predetermined pulse amplitudes, widths, frequencies, or electrode polarities for the pulses delivered for these various impedance measurements. In some examples, the amplitudes and/or widths of the pulses may be sub-threshold, e.g., below a threshold necessary to capture or otherwise activate tissue, such as cardiac tissue, subcutaneous tissue, or muscle tissue. In some examples, IMD 10 may use an amplifier circuit to perform physiological signal sensing, impedance sensing, telemetry, etc.

In certain cases, IMD 10 may measure subcutaneous impedance values that include both a resistive component and a reactive component (e.g., X, XL, XC), such as in an impedance triangle. In such cases, IMD 10 may measure subcutaneous impedance during delivery of a sinusoidal or other time varying signal by impedance measurement circuitry 60, for example. Thus, as used herein, the term “impedance” is used in a broad sense to indicate any collected, measured, and/or calculated value that may include one or both of resistive and reactive components. In some examples, subcutaneous tissue impedance parameters are derived from subcutaneous tissue impedance signals received from electrodes 16.

Sensing circuitry 52 may also provide one or more impedance signals to processing circuitry 50 for analysis, e.g., for analysis to determine respiration and impedance parameters, e.g., impedance scores. In some examples, processing circuitry 50 may store the impedance values, impedance score factors (e.g., fluid indices, average impedance values, reference impedance values, buffer values, etc.), and impedance scores in storage device 56. Processing circuitry 50 of IMD 10, and/or processing circuitry of another device that retrieves data from IMD 10, may analyze the impedance values to determine a diagnostic state of the subcutaneous tissue impedance parameter.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network.

Antenna 26 and communication circuitry 54 may be configured to transmit and/or receive signals via inductive coupling, electromagnetic coupling, NFC technologies, RF communication, Bluetooth®, Wi-Fi™, or other proprietary or non-proprietary wireless communication schemes. In some examples, processing circuitry 50 may provide data to be uplinked to external device 12 via communication circuitry 54 and control signals using an address/data bus. In another example, communication circuitry 54 may provide received data to processing circuitry 50 via a multiplexer.

In some examples, processing circuitry 50 may send impedance data to external device 12 or data server(s) 94 via communication circuitry 54. For example, IMD 10 may send external device 12 or data server(s) 94 collected impedance measurements. External device 12 and/or data server(s) 94 may then analyze those impedance measurements.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media. For example, storage device 56 may include random access memory (RAM), read-only memory (ROM), non-volatile RAM (NVRAM), electrically-erasable programmable ROM (EEPROM), erasable programmable ROM (EPROM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include impedance values and/or digitized cardiac EGMs, as examples.

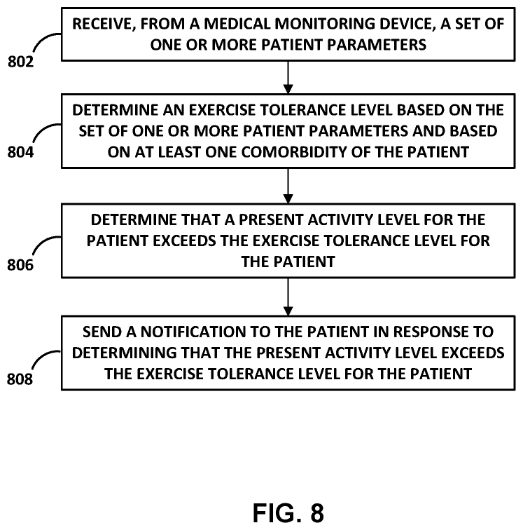

FIG. 7 is a conceptual side-view diagram illustrating an example configuration of an IMD, such as IMD 10 described with reference to FIGS. 7 and 8. The conceptual side-view diagram illustrates a muscle layer 20 and a skin layer 18 (e.g., dermis layer, epidermis layer). The region between muscle layer 20 and skin layer 18 includes subcutaneous space 22. Subcutaneous space includes blood vessels 24, such as capillaries, arteries, or veins, and interstitial fluid in the interstitium 28 of subcutaneous space 22. Subcutaneous space 22 has interstitial fluid that is commonly found between skin 18 and muscle layer 20. Subcutaneous space 22 may include interstitial fluid that surrounds blood vessels 24. For example, interstitial fluid surrounds capillaries and allows the passing of capillary elements (e.g., nutrients) between the different layers of a body through interstitium 28.

In the example shown in FIG. 7, IMD 10 may include a leadless, subcutaneously implantable monitoring device having a housing 15 and an insulative cover 76. Electrodes 16 may be formed or placed on an outer surface of cover 76. Although the illustrated example includes three electrodes 16, IMDs including or coupled to more or less than three electrodes 16 may implement the techniques of this disclosure in some examples. In some examples, electrodes 16 may be disposed all within a single layer, such as subcutaneous space 22 and contact interstitial fluid in subcutaneous space 22.

Circuitries 50-62 may be formed or placed on an inner surface of cover 76, or within housing 15. In the illustrated example, antenna 26 is formed or placed on the inner surface of cover 76, but may be formed or placed on the outer surface in some examples. In some examples, one or more of sensors 62 may be formed or placed on the outer surface of cover 76. In some examples, insulative cover 76 may be positioned over an open housing 15 such that housing 15 and cover 76 enclose antenna 26 and circuitries 50-62, and protect antenna 26 and circuitries from fluids such as interstitial fluids or other bodily fluids.

One or more of antenna 26 or circuitries 50-62 may be formed on the inner side of insulative cover 76, such as by using flip-chip technology. Insulative cover 76 may be flipped onto a housing 15. When flipped and placed onto housing 15, the components of IMD 10 formed on the inner side of insulative cover 76 may be positioned in a gap 78 defined by housing 15. Electrodes 16 may be electrically connected to switching circuitry 58 through one or more vias (not shown) formed through insulative cover 76. Insulative cover 76 may be formed of sapphire (i.e., corundum), glass, parylene, and/or any other suitable insulating material. Housing 15 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

In one example of the techniques of this disclosure, external device 12 receives, from IMD 10, a set of one or more patient parameters. External device 12 determines an exercise tolerance level based on the set of one or more patient parameters and based on at least one comorbidity of the patient. External device 12 may, for example, apply a probability model like probability model 19 or may transfer the set of one or more patient parameters to another device, such that the other device can apply a probability model like probability model 19, and provide external device 12 with the exercise tolerance level. IMD 10 and external device 12 may jointly or individually determine that a present activity level exceeds the exercise tolerance level and provide a notification to patient 4.

FIG. 8 is a flow diagram illustrating a process in accordance with the techniques described herein. The techniques of FIG. 8 will be described with respect to a medical monitoring device and one or more external computing devices. The medical monitoring device may, for example, correspond to IMD 10 or one of medical device(s) 17, and the one or more external computing devices may correspond to external device 12, data server(s) 94, computing devices 100, or any combination thereof. The one or more external computing devices receive, from a medical monitoring device, a set of one or more patient parameters (802). The set of one or more patient parameters include parameters usable to determine if the patient is engaged in a dangerous activity level in view of the comorbidity of the patient and may include any of the various parameter discussed throughout this disclosure.

The one or more external computing devices determine an exercise tolerance level based on the set of one or more patient parameters and based on at least one comorbidity of the patient (804). The one or more external computing devices may determine the exercise tolerance level based on the one or more patient parameters and one or more additional parameters that are not received from the medical monitoring device. The one or more additional parameters may, for example, include an indication of one or more comorbidities of the patient. To determine the exercise tolerance level based on the set of one or more patient parameters, the one or more external computing devices may apply the set of one or more patient parameters to a patient model that is configured at least in part based on data collected from a cohort of patients that have the at least one comorbidity in common with the patient or a patient model that is configured at least in part based on past data of the patient. The patient models may include a Bayesian framework. The patient models may weight different parameters differently.

The one or more external computing devices determine that a present activity level for the patient exceeds the exercise tolerance level for the patient (806). The one or more external computing devices may determine the present activity level for the patient based on a second set of one or more patient parameters, which may either be fully distinct from, or partially or fully overlap with, the set of one or more patient parameters received from the medical monitoring device.

The one or more external computing devices send a notification to the patient in response to determining that the present activity level exceeds the exercise tolerance level for the patient (808). The notification may, for example, include an alert that the patient is engaged in an activity detrimental to the health of the patient and may also include a recommended action for the patient to reduce a risk of a cardiac event for the patient.

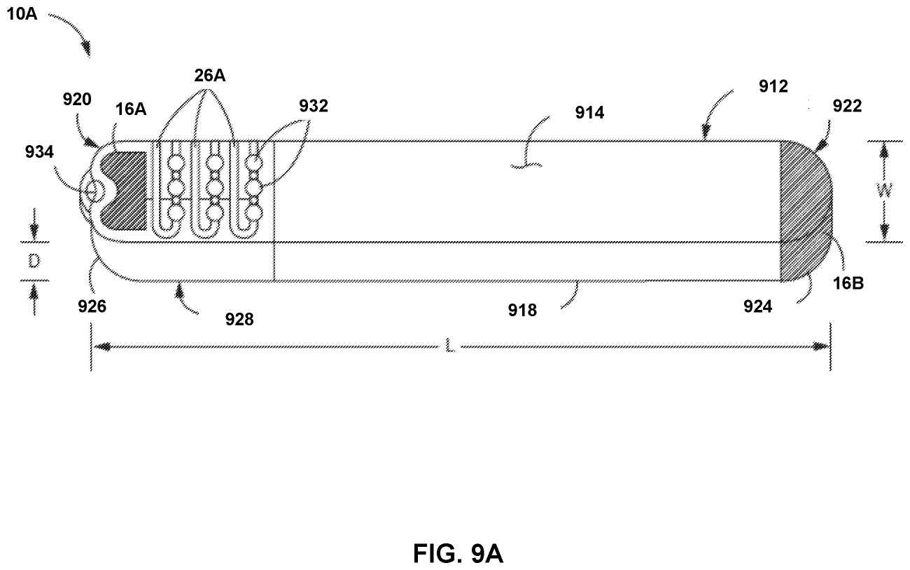

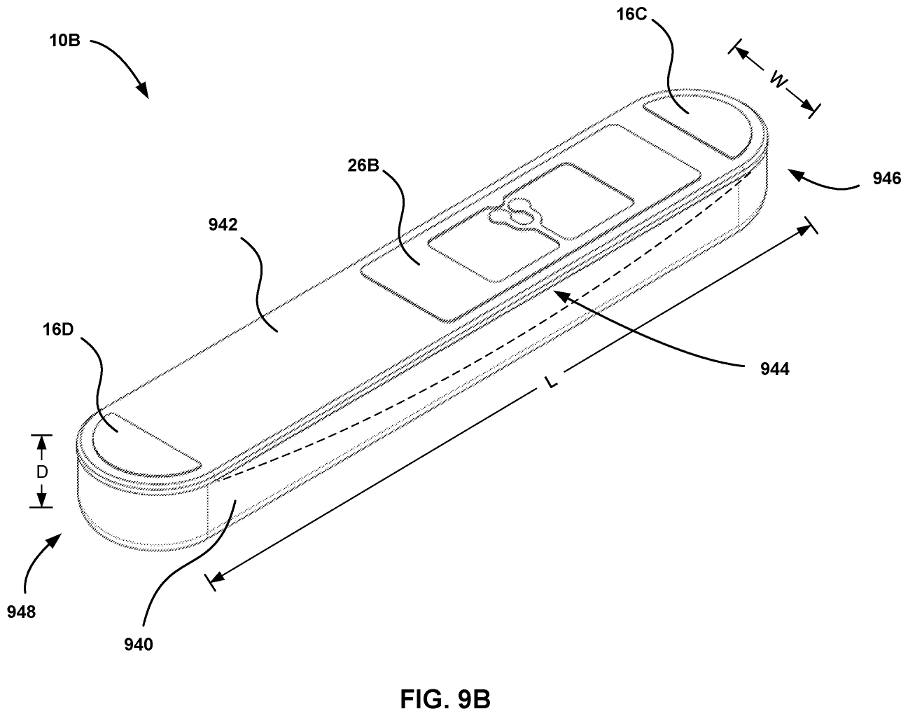

FIG. 9A is a perspective drawing illustrating an IMD 10A, which may be an example configuration of IMD 10 of FIGS. 5-7 as an ICM. In the example shown in FIG. 9A, IMD 10A may be embodied as a monitoring device having housing 912, proximal electrode 16A and distal electrode 16B. Housing 912 may further comprise first major surface 914, second major surface 918, proximal end 920, and distal end 922. Housing 912 encloses electronic circuitry located inside the IMD 10A and protects the circuitry contained therein from body fluids. Housing 912 may be hermetically sealed and configured for subcutaneous implantation. Electrical feedthroughs provide electrical connection of electrodes 16A and 16B.

In the example shown in FIG. 9A, IMD 10A is defined by a length L, a width W and thickness or depth D and is in the form of an elongated rectangular prism wherein the length L is much larger than the width W, which in turn is larger than the depth D. In one example, the geometry of the IMD 10A—in particular a width W greater than the depth D—is selected to allow IMD 10A to be inserted under the skin of the patient using a minimally invasive procedure and to remain in the desired orientation during insertion. For example, the device shown in FIG. 9A includes radial asymmetries (notably, the rectangular shape) along the longitudinal axis that maintains the device in the proper orientation following insertion. For example, the spacing between proximal electrode 16A and distal electrode 16B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 10A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 14 may range from 3 mm to 15, mm, from 3 mm to 10 mm, or from 5 mm to 15 mm, and may be any single or range of widths between 3 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 9A, once inserted within the patient, the first major surface 914 faces outward, toward the skin of the patient while the second major surface 918 is located opposite the first major surface 914. In addition, in the example shown in FIG. 9A, proximal end 920 and distal end 922 are rounded to reduce discomfort and irritation to surrounding tissue once inserted under the skin of the patient. IMD 10A, including instrument and method for inserting IMD 10A is described, for example, in U.S. Patent Publication No. 2014/0276928, incorporated herein by reference in its entirety.

Proximal electrode 16A is at or proximate to proximal end 920, and distal electrode 16B is at or proximate to distal end 922. Proximal electrode 16A and distal electrode 16B are used to sense cardiac EGM signals, e.g., ECG signals, thoracically outside the ribcage, which may be sub-muscularly or subcutaneously. EGM signals may be stored in a memory of IMD 10A, and data may be transmitted via integrated antenna 26A to another device, which may be another implantable device or an external device, such as external device 12. In some example, electrodes 16A and 16B may additionally or alternatively be used for sensing any bio-potential signal of interest, which may be, for example, an EGM, EEG, EMG, or a nerve signal, or for measuring impedance, from any implanted location. Housing 912 may house the circuitry of IMD 10 illustrated in FIG. 6.

In the example shown in FIG. 9A, proximal electrode 16A is at or in close proximity to the proximal end 920 and distal electrode 16B is at or in close proximity to distal end 922. In this example, distal electrode 16B is not limited to a flattened, outward facing surface, but may extend from first major surface 914 around rounded edges 924 and/or end surface 926 and onto the second major surface 918 so that the electrode 16B has a three-dimensional curved configuration. In some examples, electrode 16B is an uninsulated portion of a metallic, e.g., titanium, part of housing 912.

In the example shown in FIG. 9A, proximal electrode 16A is located on first major surface 914 and is substantially flat, and outward facing. However, in other examples proximal electrode 16A may utilize the three dimensional curved configuration of distal electrode 16B, providing a three dimensional proximal electrode (not shown in this example). Similarly, in other examples distal electrode 16B may utilize a substantially flat, outward facing electrode located on first major surface 914 similar to that shown with respect to proximal electrode 16A.

The various electrode configurations allow for configurations in which proximal electrode 16A and distal electrode 16B are located on both first major surface 914 and second major surface 918. In other configurations, such as that shown in FIG. 9A, only one of proximal electrode 16A and distal electrode 16B is located on both major surfaces 914 and 918, and in still other configurations both proximal electrode 16A and distal electrode 16B are located on one of the first major surface 914 or the second major surface 918 (e.g., proximal electrode 16A located on first major surface 914 while distal electrode 16B is located on second major surface 918). In another example, IMD 10A may include electrodes on both major surface 914 and 918 at or near the proximal and distal ends of the device, such that a total of four electrodes are included on IMD 10A. Electrodes 16A and 16B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 9A, proximal end 920 includes a header assembly 928 that includes one or more of proximal electrode 16A, integrated antenna 26A, anti-migration projections 932, and/or suture hole 934. Integrated antenna 26A is located on the same major surface (i.e., first major surface 914) as proximal electrode 16A and is also included as part of header assembly 928. Integrated antenna 26A allows IMD 10A to transmit and/or receive data. In other examples, integrated antenna 26A may be formed on the opposite major surface as proximal electrode 16A, or may be incorporated within the housing 912 of IMD 10A. In the example shown in FIG. 9A, anti-migration projections 932 are located adjacent to integrated antenna 26A and protrude away from first major surface 914 to prevent longitudinal movement of the device. In the example shown in FIG. 9A, anti-migration projections 932 include a plurality (e.g., nine) small bumps or protrusions extending away from first major surface 914. As discussed above, in other examples anti-migration projections 932 may be located on the opposite major surface as proximal electrode 16A and/or integrated antenna 26A. In addition, in the example shown in FIG. 9A, header assembly 928 includes suture hole 934, which provides another means of securing IMD 10A to the patient to prevent movement following insertion. In the example shown, suture hole 934 is located adjacent to proximal electrode 16A. In one example, header assembly 928 is a molded header assembly made from a polymeric or plastic material, which may be integrated or separable from the main portion of IMD 10A.

FIG. 9B is a perspective drawing illustrating another IMD 10B, which may be another example configuration of IMD 10 from FIGS. 5-7 as an ICM. IMD 10B of FIG. 9B may be configured substantially similarly to IMD 10A of FIG. 9A, with differences between them discussed herein.

IMD 10B may include a leadless, subcutaneously-implantable monitoring device, e.g. an ICM. IMD 10B includes housing having a base 940 and an insulative cover 942. Proximal electrode 16C and distal electrode 16D may be formed or placed on an outer surface of cover 942. Various circuitries and components of IMD 10B, e.g., described with respect to FIG. 6, may be formed or placed on an inner surface of cover 942, or within base 940. In some examples, a battery or other power source of IMD 10B may be included within base 940. In the illustrated example, antenna 26B is formed or placed on the outer surface of cover 942, but may be formed or placed on the inner surface in some examples. In some examples, insulative cover 942 may be positioned over an open base 940 such that base 940 and cover 942 enclose the circuitries and other components and protect them from fluids such as body fluids. The housing including base 940 and insulative cover 942 may be hermetically sealed and configured for subcutaneous implantation.

Circuitries and components may be formed on the inner side of insulative cover 942, such as by using flip-chip technology. Insulative cover 942 may be flipped onto a base 940. When flipped and placed onto base 940, the components of IMD 10B formed on the inner side of insulative cover 942 may be positioned in a gap 944 defined by base 940. Electrodes 16C and 16D and antenna 26B may be electrically connected to circuitry formed on the inner side of insulative cover 942 through one or more vias (not shown) formed through insulative cover 942. Insulative cover 942 may be formed of sapphire (i.e., corundum), glass, parylene, and/or any other suitable insulating material. Base 940 may be formed from titanium or any other suitable material (e.g., a biocompatible material). Electrodes 16C and 16D may be formed from any of stainless steel, titanium, platinum, iridium, or alloys thereof. In addition, electrodes 16C and 16D may be coated with a material such as titanium nitride or fractal titanium nitride, although other suitable materials and coatings for such electrodes may be used.

In the example shown in FIG. 9B, the housing of IMD 10B defines a length L, a width W and thickness or depth D and is in the form of an elongated rectangular prism wherein the length L is much larger than the width W, which in turn is larger than the depth D, similar to IMD 10A of FIG. 9A. For example, the spacing between proximal electrode 16C and distal electrode 16D may range from 5 mm to 50 mm, from 30 mm to 50 mm, from 35 mm to 45 mm, and may be any single spacing or range of spacings from 5 mm to 50 mm, such as approximately 40 mm. In addition, IMD 10B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 10B may have a volume of three cubic centimeters (cm) or less, or 1.5 cubic cm or less, such as approximately 1.4 cubic cm.

In the example shown in FIG. 9B, once inserted subcutaneously within the patient, outer surface of cover 942 faces outward, toward the skin of the patient. In addition, as shown in FIG. 9B, proximal end 946 and distal end 948 are rounded to reduce discomfort and irritation to surrounding tissue once inserted under the skin of the patient. In addition, edges of IMD 10B may be rounded.

The following numbered clauses illustrate one or more aspects of the devices and techniques described in this disclosure.
Clause 1. A device comprising: a memory; and processing circuitry coupled to the memory and configured to: receive, from a medical monitoring device, a set of one or more patient parameters; determine an exercise tolerance level based on the set of one or more patient parameters and based on at least one comorbidity of the patient; determine that a present activity level for the patient exceeds the exercise tolerance level for the patient; and send a notification to the patient in response to determining that the present activity level exceeds the exercise tolerance level for the patient.
Clause 2. The device of clause 1, wherein the processing circuitry is further configured to: determine the exercise tolerance level based on the one or more patient parameters and one or more additional parameters, wherein the one or more additional parameters are not received from the medical monitoring device.
Clause 3. The device of clause 1 or 2, wherein at least one of the one or more additional parameters include the comorbidity of the patient.
Clause 4. The device of any of clauses 1-3, wherein the set of one or more patient parameters comprise parameters usable to determine if the patient is engaged in a dangerous activity level in view of the comorbidity of the patient.
Clause 5. The device of any of clauses 1-4, wherein to determine the exercise tolerance level based on the set of one or more patient parameters, the processing circuitry is further configured to apply the set of one or more patient parameters to a patient model that is configured at least in part based on data collected from a cohort of patients that have the at least one comorbidity in common with the patient.
Clause 6. The device of any of clauses 1-5, wherein to determine the exercise tolerance level based on the set of one or more patient parameters, the processing circuitry is further configured to apply the set of one or more patient parameters to a patient model that is configured at least in part based on past data of the patient.
Clause 7. The device of any of clauses 1-6, wherein the processing circuitry is further configured to: determine the present activity level for the patient based on a second set of one or more patient parameters.
Clause 8. The device of clause 7, wherein the second set of one or more patient parameters are fully distinct from the set of one or more patient parameters.
Clause 9. The device of clause 7, wherein the second set of one or more patient parameters fully overlap with the set of one or more patient parameters.
Clause 10. The device of clause 7, wherein the second set of one or more patient parameters partially overlap with the set of one or more patient parameters.
Clause 11. The device of any of clauses 1-10, wherein to determine the exercise tolerance level based on the set of one or more patient parameters, the processing circuitry is further configured to: transmit the set of one or more patient parameters to an external processing system; and receive, from the external processing system, the exercise tolerance level.
Clause 12. The device of any of clauses 1-11, wherein to determine the exercise tolerance level based on the set of one or more patient parameters, the processing circuitry is further configured to determine the exercise tolerance by inputting the set of one or more patient parameters into a Bayesian framework.
Clause 13. The device of any of clauses 1-12, wherein to determine the exercise tolerance level based on the set of one or more patient parameters, the processing circuitry is further configured to apply different weightings to different patient parameters of the set of one or more patient parameters.
Clause 14. The device of any of clauses 1-13, wherein the set of one or more patient parameters includes one or more of: whether the patient has coronary artery disease; whether the patient has had a prior post myocardial infarction; a heart rate of the patient; a respiration rate of the patient; a respiration volume of the patient; an oxygen saturation level of the patient; a pulse wave transmit time of the patient; a measure of muscle activity of the patient; an ST segment deviation level for the patient; a level of T-wave alternans for the patient; and an activity level of the patient.
Clause 15. The device of any of clauses 1-13, wherein the set of one or more patient parameters includes three or more of: whether the patient has coronary artery disease; whether the patient has had a prior post myocardial infarction; a heart rate of the patient; a respiration rate of the patient; a respiration volume of the patient; an oxygen saturation level of the patient; a pulse wave transmit time of the patient; a measure of muscle activity of the patient; an ST segment deviation level for the patient; a level of T-wave alternans for the patient; and an activity level of the patient.
Clause 16. The device of any of clauses 1-13, wherein the set of one or more patient parameters includes five or more of: whether the patient has coronary artery disease; whether the patient has had a prior post myocardial infarction; a heart rate of the patient; a respiration rate of the patient; a respiration volume of the patient; an oxygen saturation level of the patient; a pulse wave transmit time of the patient; a measure of muscle activity of the patient; an ST segment deviation level for the patient; a level of T-wave alternans for the patient; and an activity level of the patient.
Clause 17. The device of any of clauses 1-16, wherein the notification comprises an alert that the patient is engaged in an activity detrimental to health of the patient.
Clause 18. The device of any of clauses 1-17, wherein the notification comprises a recommended action for the patient to reduce a risk of a cardiac event for the patient.
Clause 19. The device of any of clauses 1-18, wherein the device comprises a smartphone.
Clause 20. The device of any of clauses 1-19, wherein the medical monitoring device comprises an implantable cardiac monitor.
Clause 21. A device comprising: a memory; and processing circuitry coupled to the memory and configured to: receive, from a medical monitoring device, a set of one or more patient parameters; determine an exercise tolerance level based on the set of one or more patient parameters and based on at least one comorbidity of the patient; and transmit the exercise tolerance level to a patient device.
Clause 22. The device of clause 21, wherein the processing circuitry is further configured to: determine the exercise tolerance level based on the one or more patient parameters and one or more additional parameters, wherein the one or more additional parameters are not received from the medical monitoring device.
Clause 23. The device of clause 21 or 22, wherein at least one of the one or more additional parameters include the comorbidity of the patient.
Clause 24. The device of any of clauses 21-23, wherein the set of one or more patient parameters comprise parameters usable to determine if the patient is engaged in a dangerous activity level in view of the comorbidity of the patient.
Clause 25. The device of any of clauses 21-24, wherein to determine the exercise tolerance level based on the set of one or more patient parameters, the processing circuitry is further configured to apply the set of one or more patient parameters to a patient model that is configured at least in part based on data collected from a cohort of patients that have the at least one comorbidity in common with the patient.
Clause 26. The device of any of clauses 21-25, wherein to determine the exercise tolerance level based on the set of one or more patient parameters, the processing circuitry is further configured to apply the set of one or more patient parameters to a patient model that is configured at least in part based on past data of the patient.
Clause 27. The device of any of clauses 21-26, wherein the processing circuitry is further configured to: determine the present activity level for the patient based on a second set of one or more patient parameters.
Clause 28. The device of clause 27, wherein the second set of one or more patient parameters are fully distinct from the set of one or more patient parameters.
Clause 29. The device of clause 27, wherein the second set of one or more patient parameters fully overlap with the set of one or more patient parameters.
Clause 30. The device of clause 27, wherein the second set of one or more patient parameters partially overlap with the set of one or more patient parameters.
Clause 31. The device of any of clauses 21-30, wherein to determine the exercise tolerance level based on the set of one or more patient parameters, the processing circuitry is further configured to determine the exercise tolerance by inputting the set of one or more patient parameters into a Bayesian framework.
Clause 32. The device of any of clauses 21-31, wherein to determine the exercise tolerance level based on the set of one or more patient parameters, the processing circuitry is further configured to apply different weightings to different patient parameters of the set of one or more patient parameters.
Clause 33. The device of any of clauses 21-32, wherein the device comprises a cloud-based device.
Clause 34. The device of any of clauses 21-33, wherein the medical monitoring device comprises an implantable cardiac monitor.
Clause 35. A method comprising: receiving, from a medical monitoring device, a set of one or more patient parameters; determining an exercise tolerance level based on the set of one or more patient parameters and based on at least one comorbidity of the patient; determining that a present activity level for the patient exceeds the exercise tolerance level for the patient; and sending a notification to the patient in response to determining that the present activity level exceeds the exercise tolerance level for the patient.
Clause 36. The method of clause 35, further comprising: determining the exercise tolerance level based on the one or more patient parameters and one or more additional parameters, wherein the one or more additional parameters are not received from the medical monitoring device.
Clause 37. The method of clause 35 or 36, wherein at least one of the one or more additional parameters include the comorbidity of the patient.
Clause 38. The method of any of clauses 35-37, wherein the set of one or more patient parameters comprise parameters usable to determine if the patient is engaged in a dangerous activity level in view of the comorbidity of the patient.
Clause 39. The method of any of clauses 35-38, wherein determining the exercise tolerance level based on the set of one or more patient parameters comprises applying the set of one or more patient parameters to a patient model that is configured at least in part based on data collected from a cohort of patients that have the at least one comorbidity in common with the patient.
Clause 40. The method of any of clauses 35-39, wherein determining the exercise tolerance level based on the set of one or more patient parameters comprises applying the set of one or more patient parameters to a patient model that is configured at least in part based on past data of the patient.
Clause 41. The method of any of clauses 35-40, further comprising: determining the present activity level for the patient based on a second set of one or more patient parameters.
Clause 42. The method of clause 41, wherein the second set of one or more patient parameters are fully distinct from the set of one or more patient parameters.
Clause 43. The method of clause 41, wherein the second set of one or more patient parameters fully overlap with the set of one or more patient parameters.
Clause 44. The method of clause 41, wherein the second set of one or more patient parameters partially overlap with the set of one or more patient parameters.
Clause 45. The method of any of clauses 35-44, wherein determining the exercise tolerance level based on the set of one or more patient parameters comprises: transmit the set of one or more patient parameters to an external processing system; and receive, from the external processing system, the exercise tolerance level.
Clause 46. The method of any of clauses 35-45, wherein determining the exercise tolerance level based on the set of one or more patient parameters comprises determining the exercise tolerance by inputting the set of one or more patient parameters into a Bayesian framework.
Clause 47. The method of any of clauses 35-46, wherein determining the exercise tolerance level based on the set of one or more patient parameters comprises applying different weightings to different patient parameters of the set of one or more patient parameters.

The techniques described in this disclosure may be implemented, at least in part, in hardware, software, firmware, or any combination thereof. For example, various aspects of the techniques may be implemented within one or more microprocessors, DSPs, ASICS, FPGAs, or any other equivalent integrated or discrete logic QRS circuitry (as in QRS complex), as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, ROM, NVRAM, DRAM, SRAM, Flash memory, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD or other medical device, an external programmer, a combination of a medical device and external programmer, an integrated circuit (IC) or a set of ICs, and/or discrete electrical circuitry, residing in a medical device and/or external programmer.

Furthermore, although described primarily with reference to examples that provide a probability score to indicate worsening heart failure, other examples may additionally or alternatively automatically modify a therapy in response to the probability score exceeding a predetermined threshold. The therapy may be, as examples, a substance delivered by an implantable pump, cardiac resynchronization therapy, refractory period stimulation, or cardiac potentiation therapy. These and other examples are within the scope of the following claims.

1. A medical device system comprising:
an implantable or wearable medical device configured to determine values of a set of one or more patient parameters based on one or more sensed patient signals; and
processing circuitry configured to:
determine an exercise tolerance level for the patient based on the values of the set of one or more patient parameters and based on at least one comorbidity of the patient;
determine that a present activity level for the patient exceeds the exercise tolerance level for the patient; and
send a notification to the patient in response to determining that the present activity level exceeds the exercise tolerance level for the patient.

2. The system of claim 1, wherein the processing circuitry is further configured to:
determine the exercise tolerance level based on the values of the set of one or more patient parameters and values of one or more additional parameters, wherein the values of the one or more additional parameters are not received from the medical device.

3. The system of claim 2, wherein at least one of the one or more additional parameters includes a parameter indicative of the comorbidity of the patient.

4. The system of claim 1, wherein the set of one or more patient parameters comprise parameters usable to determine if the patient is engaged in a dangerous activity level in view of the comorbidity of the patient.

5. The system of claim 1, wherein to determine the exercise tolerance level based on the values of the set of one or more patient parameters, the processing circuitry is further configured to apply values of the set of one or more patient parameters to a patient model that is configured at least in part based on data collected from a cohort of patients that have the at least one comorbidity in common with the patient.

6. The system of claim 1, wherein to determine the exercise tolerance level based on the values of the set of one or more patient parameters, the processing circuitry is further configured to apply the values of the set of one or more patient parameters to a patient model that is configured at least in part based on past data of the patient.

7. The system of claim 1, wherein the processing circuitry is further configured to:
determine the present activity level for the patient based on values of a second set of one or more patient parameters.

8. The system of claim 7, wherein the second set of one or more patient parameters are fully distinct from or fully overlap with the set of one or more patient parameters.

9. The system of claim 1, wherein the processing circuitry comprises processing circuitry of at least one of a smartphone of the patient or a cloud computing system configured to determine the exercise tolerance level based on the values of the set of one or more patient parameters.

10. The system of claim 1, wherein the processing circuitry is configured to determine the exercise tolerance by inputting the values of the set of one or more patient parameters into a Bayesian framework.

11. The system of claim 1, wherein to determine the exercise tolerance level based on the values of the set of one or more patient parameters, the processing circuitry is configured to apply different weightings to different patient parameters of the set of one or more patient parameters.

12. The system of claim 1, wherein the set of one or more patient parameters includes one or more of:
a heart rate of the patient;
a respiration rate of the patient;
a respiration volume of the patient;
an oxygen saturation level of the patient;
a pulse wave transmit time of the patient;
a measure of muscle activity of the patient;
an ST segment deviation level for the patient;
a level of T-wave alternans for the patient; and
an activity level of the patient.

13. The system of claim 1, wherein the set of one or more patient parameters includes three or more of:
a heart rate of the patient;
a respiration rate of the patient;
a respiration volume of the patient;
an oxygen saturation level of the patient;
a pulse wave transmit time of the patient;
a measure of muscle activity of the patient;
an ST segment deviation level for the patient;
a level of T-wave alternans for the patient; and
an activity level of the patient.

14. The system of claim 1, wherein the set of one or more patient parameters includes five or more of:
a heart rate of the patient;
a respiration rate of the patient;
a respiration volume of the patient;
an oxygen saturation level of the patient;
a pulse wave transmit time of the patient;
a measure of muscle activity of the patient;
an ST segment deviation level for the patient;
a level of T-wave alternans for the patient; and
an activity level of the patient.

15. The system of claim 1, wherein the at least one comorbidity comprises at least one of heart failure, coronary artery disease, or prior myocardial infarction.

16. The system of claim 1, wherein processing circuitry is configured to:
determine a risk level of a cardiac event comprising arrhythmia, myocardial infarction, or ischemic stroke of the patient based on the values of the set of one or more patient parameters; and
determine the exercise tolerance level based on the risk level of the cardiac event.

17. The system of claim 1, wherein the processing circuitry comprises processing circuitry of at least one of the medical device or a smartphone of the patient configured to determine that the present activity level for the patient exceeds the exercise tolerance level for the patient.

18. The system of claim 1, wherein the implantable medical device comprises an insertable cardiac monitor comprising:
a housing configured for subcutaneous implantation in a patient, the housing having a length between 40 millimeters (mm) and 60 mm between a first end and a second end, a width less than the length, and a depth less than the width;
a first electrode at or proximate to the first end; and
a second electrode at or proximate to the second end,
wherein the insertable cardiac monitor is configured to sense at least one of the set of one or more patient parameters via the first electrode and the second electrode.
